(19) United States
(12) Patent Application Publication
(10) Pub. No.: US 2025/0152593 A1
(43) Pub. Date: May 15, 2025

(54) p38 MAPK INHIBITORS AND USES THEREOF

(71) Applicants: CITY OF HOPE, Duarte, CA (US); NATIONAL AND KAPODISTRIAN UNIVERSITY OF ATHENS, Athens (GR)

(72) Inventors: Sangkil Nam, Tujunga, CA (US); Hannah Zhang, Duarte, CA (US); Steven Rosen, La Quinta, CA (US); Ioannis K. Kostakis, Athens (GR); Alexios Leandros Skaltsounis, Athens (GR); Eleftheria A. Georgiou, Athens (GR)

(21) Appl. No.: 18/941,810

(22) Filed: Nov. 8, 2024

Related U.S. Application Data
(60) Provisional application No. 63/597,968, filed on Nov. 10, 2023.

(51) Int. Cl. A61K 31/519; A61K 31/167; A61K 38/15; C07D 487/04

(52) U.S. Cl. CPC A61K 31/519; A61K 31/167; A61K 38/15; C07D 487/04

(57) ABSTRACT

Described herein, inter alia, are p38 MAPK inhibitors and uses thereof.

### CROSS-REFERENCE TO RELATED APPLICATIONS

This application claims the benefit of U.S. Provisional Application No. 63/597,968 filed Nov. 10, 2023, the contents of which is hereby incorporated herein in its entirety and for all purposes.

### REFERENCE TO AN ELECTRONIC SEQUENCE LISTING

The contents of the electronic sequence listing (048440-794001US-Sequence-Listing-ST26.xml; Size: 4,226 bytes; and Date of Creation: Oct. 15, 2024) are hereby incorporated by reference in their entirety.

### BACKGROUND

p38 MAPK inhibitors show therapeutic effects on cancer, autoimmune, and inflammatory diseases, as well as chronic obstructive pulmonary disease. Disclosed herein, inter alia, are solutions to these and other problems in the art.

### BRIEF SUMMARY

In an aspect is provided a compound, or a pharmaceutically acceptable salt thereof, having the formula:

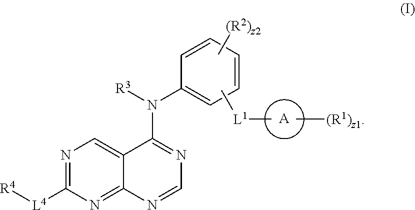

Ring A is cycloalkyl, heterocycloalkyl, aryl, or heteroaryl.

L¹ is —NR¹⁰C(O)— or —C(O)NR¹⁰—.

L⁴ is a

bond, —C(O)—, —C(O)O—, —OC(O)—, —O—, —S—, —NR⁴⁰—, —C(O)NR⁴⁰—, —NR⁴⁰C(O)—, —NR⁴⁰C(O)O—, —OC(O)NR⁴⁰—, —NR⁴⁰C(O)NR^{40A}, —S(O)₂—, —NR⁴⁰S(O)₂—, —S(O)₂NR⁴⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ is independently

halogen, —CX¹³, —CHX¹², —CH₂X¹, —OCX¹³, —OCH₂X¹, —OCHX¹², —CN, —SOₙ₁R_{1D}, —SOᵥ₁NR^{1A}R^{1B}, NR^{1C}NR^{1A}R^{1B}, ONR^{1A}R^{1B}, NR^{1C}C(O)NR^{1A}R^{1B}, N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is independently

halogen, —CX²³, —CHX²², —CH₂X², —OCX²³, —OCH₂X², —OCHX²², —CN, —SOn₂R^{2D}, —SOᵥ₂NR^{2A}R^{2B}, —NR²CNR^{2A}R^{2B}, —ONR^{2A}R^{2B}, —NR^{2C}C(O)NR^{2A}R^{2B}, —N(O)m₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is hydrogen,

halogen, —CX⁴₃, —CHX⁴₂, —CH₂X⁴, —OCX⁴₃, —OCH₂X⁴, —OCHX⁴₂, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4A}R^{4B}, —N(O)ₘ₄, —NR^{4A}R^{4B}, —C(O)R^{4C}, —C(O)OR^{4C}, —C(O)NR^{4A}R^{4B}—OR^{4D}, —SR^{4D}, —NR⁴ASO₂R^{4D}, —NR⁴AC(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁰, R⁴⁰, and R^{40A} are independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A}, R^{1B}, R^{1C}, R^{1D}, R^{2A}, R^{2B}, R^{2C}, R^{2D}, R^{4A}, R^{4B}, R^{4C}, and R^{4D} are independently

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{IB} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl.

The symbol z1 is an integer from 0 to 7. The symbol z2 is an integer from 0 to 4.

X¹, X², and X⁴ are independently —Cl, —Br, —I, or —F. The symbols n1, n2, and n4 are independently an integer from 0 to 4. The symbols m1, m2, m4, v1, v2, and v4 are independently 1 or 2.

In an aspect is provided a pharmaceutical composition including a compound described herein, or a pharmaceutically acceptable salt thereof, and a pharmaceutically acceptable excipient.

In an aspect is provided a method of treating cancer in a subject in need thereof, the method including administering to the subject a therapeutically effective amount of a compound described herein, or a pharmaceutically acceptable salt thereof.

In an aspect is provided a method of treating a p38 MAPK-associated disease in a subject in need thereof, the method including administering to the subject a therapeutically effective amount of a compound described herein, or a pharmaceutically acceptable salt thereof.

In an aspect is provided a method of modulating the level of activity of a p38 MAPK protein in a cell, the method including contacting the cell with an effective amount of a compound described herein, or a pharmaceutically acceptable salt thereof.

### BRIEF DESCRIPTION OF THE DRAWINGS

FIG. 1. Antitumor activities of select compounds against various cancer cell lines.

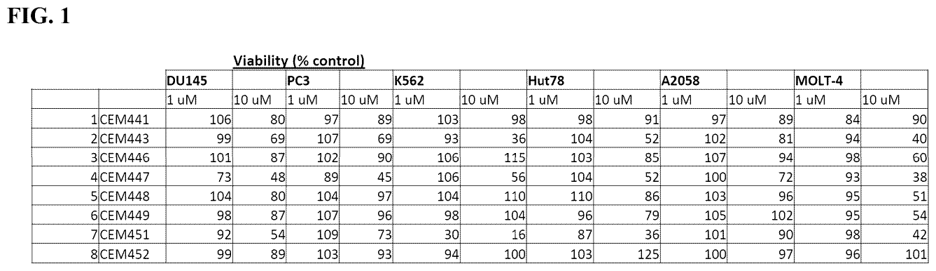

FIG. 2. CEM451 is a dual inhibitor of p38a and p38p kinases.

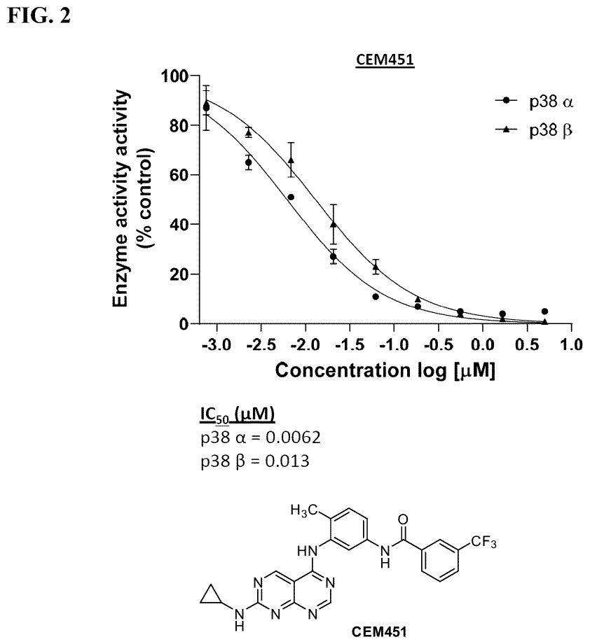

FIG. 3. Docking calculation. CEM451 interacts with the gatekeeper MET109 of p38a. Glide score: −9.7. Sequences shown: Val30; Val38-Ala40; Ala51-Val52-Lys53; Glu71-Leu74-Leu75-Met78; Val83-Ile84; Leu104-Val105-Thr106-His107-Leu108-Met109-Gly110 (residues 104.110 of SEQ ID NO:1); His148; Ile166-Leu167-Asp168-Phe169 (residues 166.169 of SEQ ID NO:1).

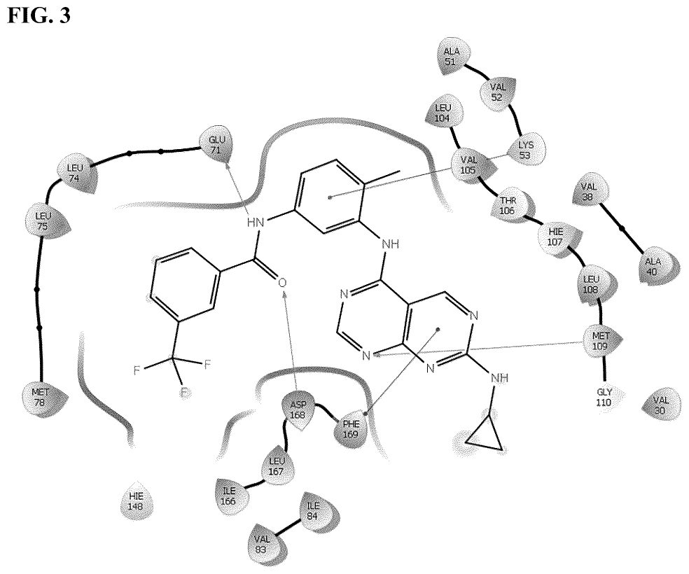

FIG. 4. Effects of CEM451 on hematopoietic cells.

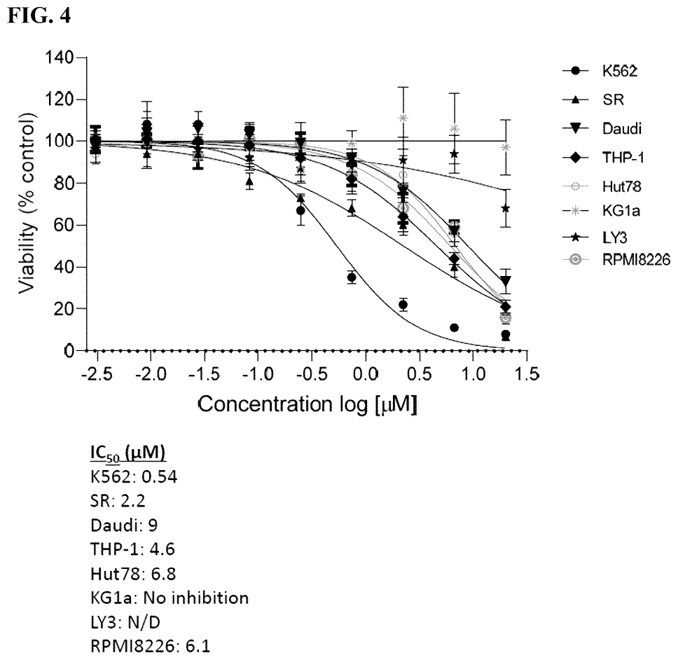

FIG. 5. Chemical structures of SAHA and romidepsin.

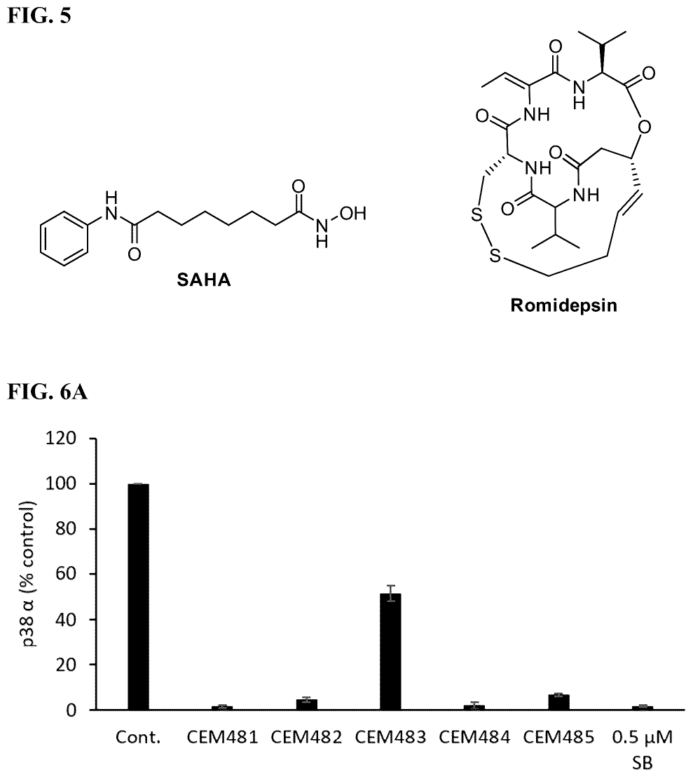

FIGS. 6A-6C. In vitro kinase assays for p38a (FIG. 6A), p38β(FIG. 6B), and p387 (FIG. 6C) at 100 nM.

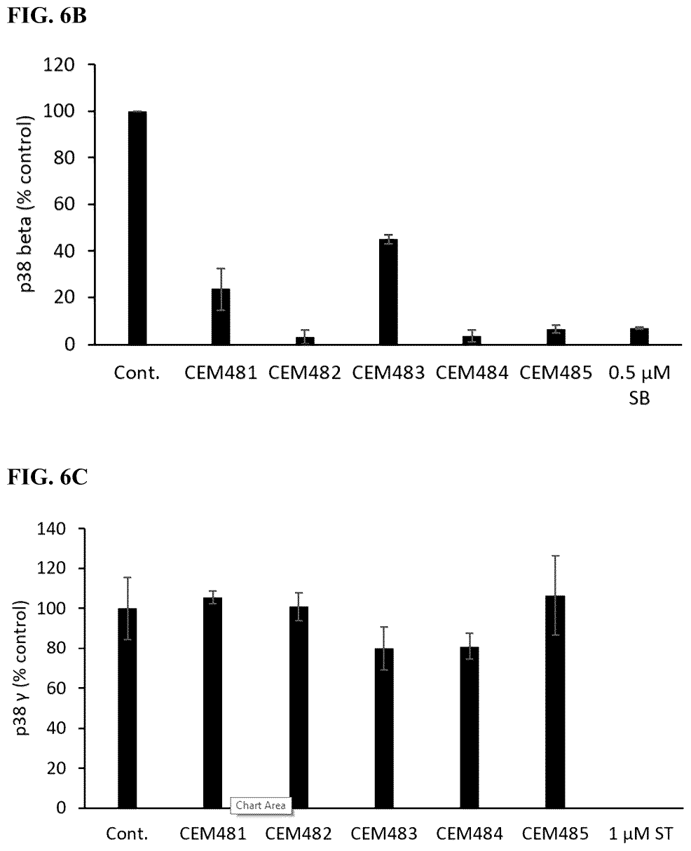

FIG. 7. Effects of CEM derivatives on human cancer cells.

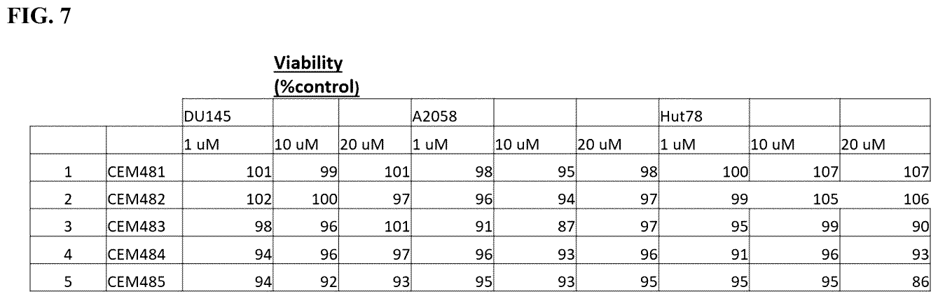

FIG. 8. Effects of CEM derivatives on Hut78 CTCL cells.

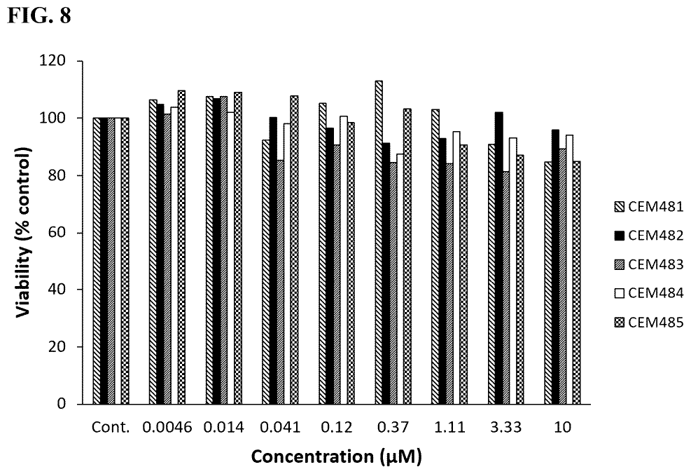

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by,

—CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to:

—CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)3, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)3. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as

—C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

[symbol]

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

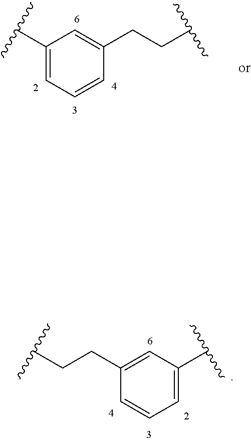 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, =N—OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′, —NR″C(O)₂R′, —NRC(NR′R″R″′)═NR″″, —NRC(NR′R″)═NR″′, —S(O)R′, —S(O)₂R′, —S(O)₂NR′R″, —NRSO₂R′, —NR′NR″R″′, —ONR′R″, —NR′C(O)NR″NR″R″′, —CN, —NO₂, —NR′SO₂R″, —NR′C(O)R″, —NR′C(O)OR″, —NR′OR″, in a number ranging from zero to (2 m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1.3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″′, —OC(O)R′, —C(O)R′, —CO₂R′, —CONR′R″, —OC(O)NR′R″, —NR″C(O)R′, —NR′C(O)NR″R″′″, —NR″C(O)2R′, —NR—C(NR′R″R′″)═NR″″, —NR—C(NR′R″)═NR″′″, —S(O)R′, —S(O)₂R′, —S(O)₂NR′R″, —NRSO₂R′, —NR′NR″R′″, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)2-, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃,
—SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃,
   —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl,
      —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃,
      —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and/or
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A}., R^{3A} may be substituted with one or more first substituent groups denoted by R^{3A.1}, R^{4A}may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R″ or L″ wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1}, . . . , R^{100A.1}; R^{L1.1}, R^{L2.1}, R^{L3.1}, R^{L4.1} R^{L5.1}, . . . , R^{L100.1}) may be further substituted with one or more second substituent groups (e.g., R^{1.2}, R^{2.2}, R^{3.2}, R^{4.2}, R^{5.2}, . . . R^{100.2}; R^{1A.2}, R^{2A.2}, R^{3A.2}, R^{4A.2}, R^{5A.2}, . . . R^{100.2}; R^{L12}, R^{L2.2}, R^{L3.2}, R², R^{L5.2} . . . R^{L100.2} respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100.2}; R^{L.12}, R^{L2.2}, R^{L3.2}, R^{L.2}, R^{L5.2} . . . R^{L100.2}) may be further substituted with one or more third substituent groups (e.g., R^{1.3}, R^{2.3}, R^{3.3}, R^{4.2}, R^{5.2}, . . . , R^{100.3}; R^{1A.3}, R^{2A.3}, R^{3A.3}, R^{4A.3}, R^{5A.3}, . . . , R^{100A.3}; R^{L1.3}, R^{L2.3}, R^{L3.3}, R^{L4.3}, R^{L5.3} . . . R^{L100.3}. respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WWW.3}. By way of example when the R^{WW} group is phenyl substituted by R^{WW.1}, which is methyl, the methyl group may be further substituted to form groups including but not limited to:

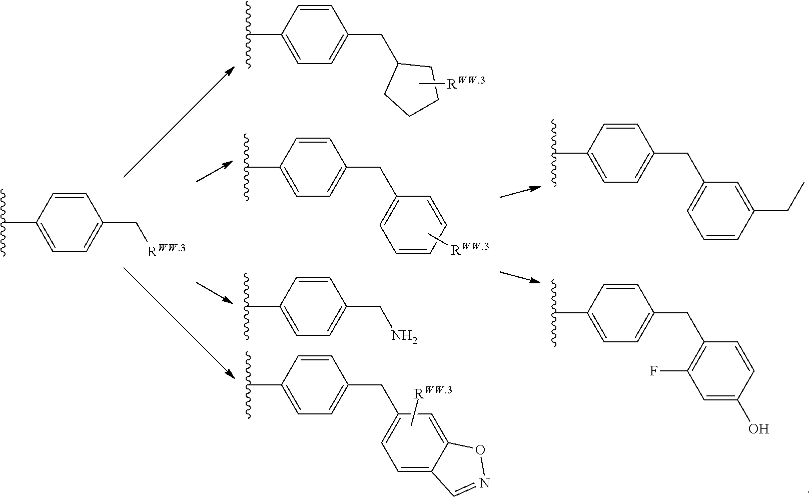

R¹ is independently oxo,

halogen, —CX^{WW.13, —CHX}^{WW.1}₂, —CH₂X^{WW.1}, —OCX^{WW.13, —OCH}₂X^{WW.1}, —OCHX^{WW.2}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.13, —CHX}^{WW.1}₂, —CH₂X^{WW.1}, —OCX^{WW.13, —OCH}₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo,

halogen, —CX^{WW.23}, —CHX^{WW.2}₂, —CH₂X^{WW.2}, —OCX^{WW.23},
—OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{W}3-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CXW²₃, —CHX^{WW.2}, —CH₂X^{WW.2}, —OCXW²₃, —OCH₂X^{WW.2}, —OCHX^{WW.2}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo,

halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2} may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R″ substituents. For example, in embodiments where R^{100A} and R^{100B} are optionally joined together to form an openly substituted ring, R^{WW.1} is R^{100A.1}, R^{WW.2} is R^{10A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo,

halogen, —CX^{LWW1.3}, —CHX^{L.1}₂—CH₂X^{LWW.1}, —OCX^{LWW.13, OCH}₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{L.2}-.substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.13, —CHX}^{LWW.1}, —CH₂X^{LWW.1} —OCX^{LWW.13, —OCH}₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or CI—C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo,

halogen, —CX^{LWW.2}₃, —CHX^{LWW.2}₂, —CH₂X^{LWW.2}, —OCX^{LWW.2}₃, —OCH₂X^{LWW.2}, OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.23}, —CHX^{LWW.2}₂, —CH₂X^{LWW.2}, —OCX^{LWW.23}, —OCH₂X^{LWW.2}, OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₅, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I. R^{LWW.3} is independently oxo,
halogen, —CX^{L.3}₃, —CHX^{LWW.3}₂, —CH₂X^{LWW.3},
—OCX^{LWW.3}₃, OCH₂X^{LWW.3}, OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R″ group) is hereby defined as independently oxo, halogen, —CX^{WW}₃, —CHX^{WW}₂, —CH₂X^{WW}, —OCX^{WW}₃, —OCH₂XWW, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L″ group) is herein defined as independently a bond, —O —, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{L}WW1, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13D}, etc., wherein each of R¹³.A, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1.19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1.3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A “synergistic amount” or “synergistically effective amount” as used herein refers to the sum of a first amount (e.g., an amount of a compound provided herein) and a second amount (e.g., a therapeutic agent) that results in a synergistic effect (i.e., an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

In embodiments, a synergistic amount may be about .1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the compound provided herein when used separately from the therapeutic agent. In embodiments, a synergistic amount may be about .1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the therapeutic agent when used separately from the compound provided herein.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments, inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

“Disease” or “condition” refers to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is cancer (e.g., lymphoma, leukemia, multiple myeloma, prostate cancer, breast cancer, bladder cancer, liver cancer, lung cancer, ovarian cancer, glioblastoma, pancreatic cancer, renal cancer, head and neck cancer, colon cancer, or melanoma).

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g., an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, autoimmune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

As used herein, the term “p38 MAPK-associated disease” refers to any disease or condition caused by aberrant activity or signaling of a p38 MAPK protein. In embodiments, the p38 MAPK-associated disease is cancer (e.g., lymphoma, leukemia, multiple myeloma, prostate cancer, breast cancer, bladder cancer, liver cancer, lung cancer, ovarian cancer, glioblastoma, pancreatic cancer, renal cancer, head and neck cancer, colon cancer, or melanoma). In embodiments, the p38 MAPK-associated disease is an autoimmune disease. In embodiments, the p38 MAPK-associated disease is an inflammatory disease. In embodiments, the p38 MAPK-associated disease is chronic obstructive pulmonary disease.

The term “drug” is used in accordance with its common meaning and refers to a substance which has a physiological effect (e.g, beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²p, ³³p, ⁴⁵Ti, ⁴⁷SC, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Ph, ¹¹¹Ag, ¹⁷⁷In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh ¹¹¹Ag, ¹¹¹In, 123I, 1241, ¹²⁵I, ¹³¹I, ¹⁴²Pr, ¹⁴³Pr, ¹⁴⁹Pm, ¹⁵³Sm, ¹⁵⁴I, ¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21.29, 42, 43, 44, or 57.71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within .5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about .001 mg/kg to about 1000 mg/kg daily. A daily dose range of about .01 mg/kg to about 500 mg/kg, or about .1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to M109 of p38α when the selected residue occupies the same essential spatial or other structural relationship as M109 of p38α. In some embodiments, where a selected protein is aligned for maximum homology with p38α, the position in the aligned selected protein aligning with M109 is said to correspond to M109. Instead of a primary sequence alignment, a three dimensional structural alignment can also be used, e.g., where the structure of the selected protein is aligned for maximum correspondence with p38a and the overall structures compared. In this case, an amino acid that occupies the same essential position as M109 in the structural model is said to correspond to the M109 residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “p38 mitogen-activated protein kinase” or “p38 MAPK” refers to one or more of the family of mitogen-activated protein kinases (e.g., p38α, p38β, p387, or p386), including homologs, isoforms, and functional fragments thereof.

The term “p38α” or “mitogen-activated protein kinase 14” or “MAPK14” refers to the protein that in humans is encoded by the MAPK14 gene. In embodiments, the p38a protein encoded by the MAPK14 gene has the amino acid sequence set forth in or corresponding to Entrez 1432, UniProt Q16539, RefSeq (protein) NP-001306.1, RefSeq (protein) NP-620581.1, RefSeq (protein) NP-620582.1, or RefSeq (protein) NP-620583.1, or homolog thereof. In embodiments, the amino acid sequence or nucleic acid sequence is the sequence known at the time of filing of the present application. In embodiments, p38a has the following amino acid sequence:

(SEQ ID NO: 1)
MSQERPTFYRQELNKTIWEVPERYQNLSPVGSGAYGSVCAAFDTKTGLR
VAVKKLSRPFQSIIHAKRTYRELRLLKHMKHENVIGLLDVFTPARSLEE
FNDVYLVTHLMGADLNNIVKCQKLTDDHVQFLIYQILRGLKYIHSADII
HRDLKPSNLAVNEDCELKILDFGLARHTDDEMTGYVATRWYRAPEIMLN
WMHYNQTVDIWSVGCIMAELLTGRTLFPGTDHIDQLKLILRLVGTPGAE
LLKKISSESARNYIQSLTQMPKMNFANVFIGANPLAVDLLEKMLVLDSD
KRITAAQALAHAYFAQYHDPDDEPVADPYDQSFESRDLLIDEWKSLTYD
EVISFVPPPLDQEEMES.

The term “p38β” or “mitogen-activated protein kinase 11” or “MAPK11” refers to the protein that in humans is encoded by the MAPK11 gene. In embodiments, the p38βprotein encoded by the MAPK11 gene has the amino acid sequence set forth in or corresponding to Entrez 5600, UniProt Q15759, or RefSeq (protein) NP-002742.3, or homolog thereof. In embodiments, the amino acid sequence or nucleic acid sequence is the sequence known at the time of filing of the present application. In embodiments, p38βhas the following amino acid sequence:

(SEQ ID NO: 2)
MSGPRAGFYRQELNKTVWEVPQRLQGLRPVGSGAYGSVCSAYDARLRQK
VAVKKLSRPFQSLIHARRTYRELRLLKHLKHENVIGLLDVFTPATSIED
FSEVYLVTTLMGADLNNIVKCQALSDEHVQFLVYQLLRGLKYIHSAGII
HRDLKPSNVAVNEDCELRILDFGLARQADEEMTGYVATRWYRAPEIMLN
WMHYNQTVDIWSVGCIMAELLQGKALFPGSDYIDQLKRIMEVVGTPSPE
VLAKISSEHARTYIQSLPPMPQKDLSSIFRGANPLAIDLLGRMLVLDSD
QRVSAAEALAHAYFSQYHDPEDEPEAEPYDESVEAKERTLEEWKELTYQ
EVLSFKPPEPPKPPGSLEIEQ.

The term “selective” or “selectivity” or the like in reference to a compound or agent refers to the compound's or agent's ability to cause an increase or decrease in activity of a particular molecular target (e.g., protein, enzyme, etc.) preferentially over one or more different molecular targets (e.g., a compound having selectivity toward p38α or p38β would preferentially inhibit p38α or p38βover other MAPK proteins (e.g., p387 or p386)). In embodiments, a “p38α-selective compound” refers to a compound (e.g., compound described herein) having selectivity towards p38α. In embodiments, a “p38β-selective compound” refers to a compound (e.g., compound described herein) having selectivity towards p38β.

### II. Compounds

In an aspect is provided a compound, or a pharmaceutically acceptable salt thereof, having the formula:

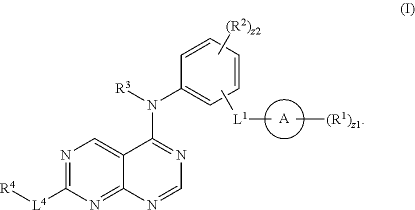

Ring A is cycloalkyl (e.g., C₃-C₈, C₃-C₆, C₄-C₆, or C₅-C₆), heterocycloalkyl (e.g., 3 to 8 membered, 3 to 6 membered, 4 to 6 membered, 4 to 5 membered, or 5 to 6 membered), aryl (e.g., C₆-C₁₀ or phenyl), or heteroaryl (e.g., 5 to 10 membered, 5 to 9 membered, or 5 to 6 membered).

L¹ is —NR¹⁰C(O)— or —C(O)NR¹⁰—.

L⁴ is a

bond, —C(O)—, —C(O)O—, —OC(O)—, —O—, —S—, —NR⁴⁰—, —C(O)NR⁴⁰—, —NR⁴⁰C(O)—, —NR⁴⁰C(O)O—, —OC(O)NR⁴⁰—, —NR⁴⁰C(O)NR⁴⁰A-S(O)₂—, —NR⁴⁰S(O)₂—, —S(O)₂NR⁴⁰—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹ is independently

halogen, —CX¹₃, —CHX¹², —CH₂X¹, —OCX¹₃, —OCH₂X¹, —OCHX¹₂,
—CN, —SOn₁R^{1D}-SOvINR^{1A}R^{1B}, NRicNR^{1A}R^{1B}—ONR^{1A}R^{1B}—NR^{1C}C(O)NR^{1A}R^{1B}—N(O)ₘ₁, —NR^{1A}R^{1B}—C(O)R^{1C}, —C(O)OR^{1C}, —OC(O)R^{1C}, —OC(O)OR^{1C}, —C(O)NR^{1A}R^{1B}—OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² is independently

halogen, —CX²₃, —CHX²₂, —CH₂X², —OCX²₃, —OCH₂X², —OCHX²₂, —CN, —SOn₂R²D-SOᵥ₂NR^{2A}R^{2B}, —NR²cNR^{2A}R^{2B}, —ONR^{2A}R^{2B}, —NR^{2C}C(O)NR^{2A}R^{2B}, —N(O)m₂, —NR^{2A}R^{2B}, —C(O)R^{2C}, —C(O)OR^{2C}, —OC(O)R^{2C}, —OC(O)OR^{2C}, —C(O)NR^{2A}R^{2B}, —OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁-C₅, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is hydrogen, —CCl₃, —CBr₃, —CF₃, —CI₃, —CH₂Cl, —CH₂Br, —CH₂F, —CH₂I, —CHC1₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHC1₂, —OCHBr₂, —OCHF₂, —OCHI₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁴ is hydrogen,

halogen, —CX⁴₃, —CHX⁴₂, —CH₂X⁴, —OCX⁴₃, —OCH₂X⁴, —OCHX⁴₂,
—CN, —SOn₄R⁴D, —SO₄NR^{4A}R^{4B}, NR⁴cNR^{4A}R^{4B}—ONR^{4A}R^{4B}, NHC(O)NR^{4A}R^{4B}, N(O)ₘ₄, —NR^{4A}R^{4B}, —C(O)R⁴C, —C(O)OR⁴C, —C(O)NR^{4A}R^{4B}, —OR^{4D}, —SR^{4D}, —NR⁴ASO₂R⁴D, —NR⁴AC(O)R^{4C}, —NR^{4A}C(O)OR^{4C}, —NR^{4A}OR^{4C}, —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R¹⁰, R⁴⁰, and R^{40A} are independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈, C₁-C₆, C₁-C₄, or C₁-C) substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, R^{1D}, R^{2A}, R^{2B}, R^{2C}, R^{2D}, R^{4A}, R^{4B}, R^{4C}, and R^{4D} are independently

hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₅, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

The symbol z1 is an integer from 0 to 7.

The symbol z2 is an integer from 0 to 4.

X¹, X², and X⁴ are independently —Cl, —Br, —I, or —F.

The symbols n1, n2, and n4 are independently an integer from 0 to 4.

The symbols m1, m2, m4, v1, v2, and v4 are independently 1 or 2.

In embodiments, the compound has the formula:

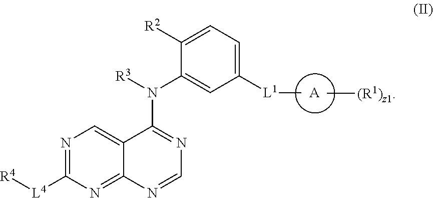

Ring A, L, R¹, z1, R², R³, L⁴, and R⁴ are as described herein, including in embodiments.

In embodiments, the compound has the formula:

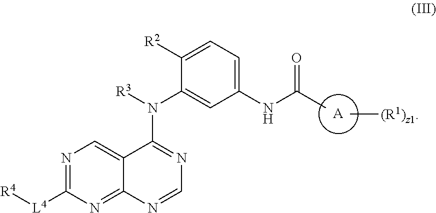

Ring A, R¹, z1, R², R³, L⁴, and R⁴ are as described herein, including in embodiments.

In embodiments, the compound has the formula:

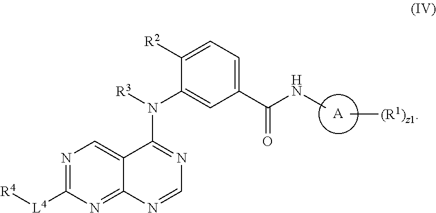

Ring A, R, z1, R², R³, L⁴, and R⁴ are as described herein, including in embodiments.

In embodiments, Ring A is cycloalkyl (e.g., C₃-C₈, C₃-C₆, C₄-C₆, or C₅-C₆). In embodiments, Ring A is heterocycloalkyl (e.g., 3 to 8 membered, 3 to 6 membered, 4 to 6 membered, 4 to 5 membered, or 5 to 6 membered). In embodiments, Ring A is aryl (e.g., C₆-C₁₀ or phenyl). In embodiments, Ring A is heteroaryl (e.g., 5 to 10 membered, 5 to 9 membered, or 5 to 6 membered). In embodiments, Ring A is C₃-C₅ cycloalkyl. In embodiments, Ring A is 3 to 8 membered heterocycloalkyl. In embodiments, Ring A is phenyl or 5 to 6 membered heteroaryl. In embodiments, Ring A is phenyl. In embodiments, Ring A is 5 to 6 membered heteroaryl. In embodiments, Ring A is pyridinyl.

In embodiments, L¹ is —NR¹⁰C(O)—. In embodiments, L¹ is —NHC(O)—. In embodiments, L¹ is —C(O)NR¹⁰—. In embodiments, L¹ is —C(O)NH—.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen, —CF₃, —CHF₂, —CH₂F, —NH₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R¹ is independently —CF₃, —N(CH₃)₂, or unsubstituted morpholinyl.

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —CCl₃. In embodiments, R¹ is independently —CBr₃. In embodiments, R¹ is independently —CF₃. In embodiments, R¹ is independently —CI₃. In embodiments, R¹ is independently —CH₂Cl. In embodiments, R¹ is independently —CH₂Br. In embodiments, R¹ is independently —CH₂F. In embodiments, R¹ is independently —CH₂I. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently —SO₃H. In embodiments, R¹ is independently —OSO₃H. In embodiments, R¹ is independently —SO₂NH₂. In embodiments, R¹ is independently —NHNH₂. In embodiments, R¹ is independently —ONH₂. In embodiments, R¹ is independently —NHC(O)NH₂. In embodiments, R¹ is independently —NHSO₂H. In embodiments, R¹ is independently —NHC(O)H. In embodiments, R¹ is independently —NHC(O)OH. In embodiments, R¹ is independently —NHOH. In embodiments, R¹ is independently —OCCl₃. In embodiments, R¹ is independently —OCBr₃. In embodiments, R¹ is independently —OCF₃. In embodiments, R¹ is independently —OCl₃. In embodiments, R¹ is independently —OCH₂Cl. In embodiments, R¹ is independently —OCH₂Br. In embodiments, R¹ is independently —OCH₂F. In embodiments, R¹ is independently —OCH₂I. In embodiments, R¹ is independently —OCHCl₂. In embodiments, R¹ is independently —OCHBr₂. In embodiments, R¹ is independently —OCHF₂. In embodiments, R¹ is independently —OCHI₂. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted 2 to 4 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy. In embodiments, R¹ is independently unsubstituted n-butoxy. In embodiments, R¹ is independently unsubstituted isobutoxy. In embodiments, R¹ is independently unsubstituted tert-butoxy. In embodiments, R¹ is independently —N(CH₃)₂. In embodiments, R¹ is independently unsubstituted 3 to 8 membered heterocycloalkyl. In embodiments, R¹ is independently unsubstituted morpholinyl.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when RA is substituted, it is substituted with at least one lower substituent group.

In embodiments, a substituted RB (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when RB is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted RD (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when RD is substituted, it is substituted with at least one lower substituent group.

In embodiments, R^{1A} is independently hydrogen. In embodiments, R^{1A} is independently unsubstituted C₁-C₄ alkyl. In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted n-propyl. In embodiments, R^{1A} is independently unsubstituted isopropyl. In embodiments, R^{1A} is independently unsubstituted butyl. In embodiments, R^{1A} is independently unsubstituted n-butyl. In embodiments, R^{1A} is independently unsubstituted isobutyl. In embodiments, R^{1A} is independently unsubstituted tert-butyl.

In embodiments, R^{1B} is independently hydrogen. In embodiments, R^{1B} is independently unsubstituted C₁-C₄ alkyl. In embodiments, R^{1B} is independently unsubstituted methyl. In embodiments, R^{1B} is independently unsubstituted ethyl. In embodiments, R^{1B} is independently unsubstituted propyl. In embodiments, RB is independently unsubstituted n-propyl. In embodiments, R^{1B} is independently unsubstituted isopropyl. In embodiments, R^{1B} is independently unsubstituted butyl. In embodiments, RB is independently unsubstituted n-butyl. In embodiments, R^{1B} is independently unsubstituted isobutyl. In embodiments, R^{1B} is independently unsubstituted tert-butyl.

In embodiments, R^{1C} is independently hydrogen. In embodiments, R^{1C} is independently unsubstituted C₁-C₄ alkyl. In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted n-propyl. In embodiments, R^{1C} is independently unsubstituted isopropyl. In embodiments, R^{1C} is independently unsubstituted butyl. In embodiments, R^{1C} is independently unsubstituted n-butyl. In embodiments, R^{1C} is independently unsubstituted isobutyl. In embodiments, R^{1C} is independently unsubstituted tert-butyl.

In embodiments, R^{1D} is independently hydrogen. In embodiments, R^{1D} is independently unsubstituted C₁-C₄ alkyl. In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted n-propyl. In embodiments, R^{1D} is independently unsubstituted isopropyl. In embodiments, R^{1D} is independently unsubstituted butyl. In embodiments, R^{1D} is independently unsubstituted n-butyl. In embodiments, R^{1D} is independently unsubstituted isobutyl. In embodiments, R^{1D} is independently unsubstituted tert-butyl.

In embodiments, z1 is 0. In embodiments, z1 is 1. In embodiments, z1 is 2. In embodiments, z1 is 3. In embodiments, z1 is 4. In embodiments, z1 is 5. In embodiments, z1 is 6. In embodiments, z1 is 7.

In embodiments,

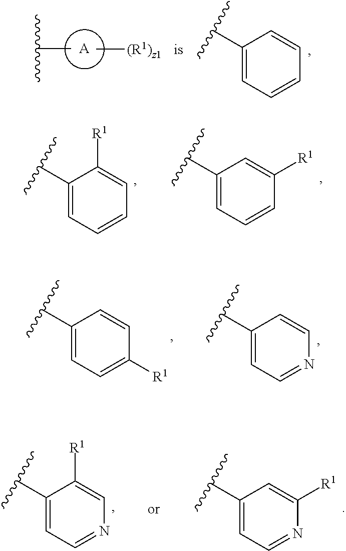

In embodiments

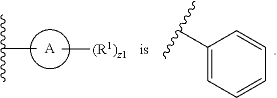

In embodiments

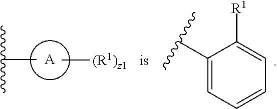

In embodiments,

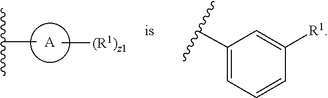

In embodiments

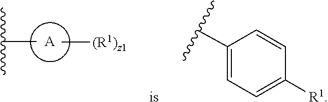

In embodiments,

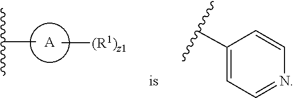

In embodiments,

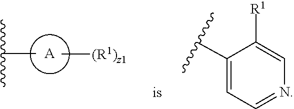

In embodiments,

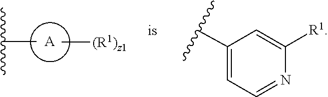

In embodiments,

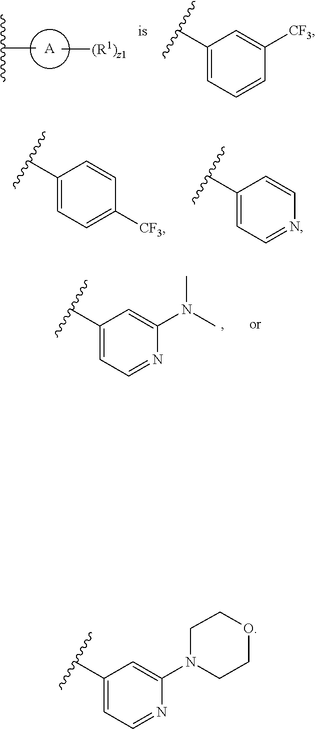

In embodiments,

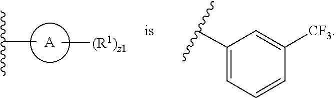

In embodiments,

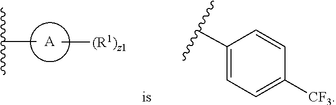

In embodiments,

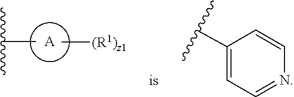

In embodiments,

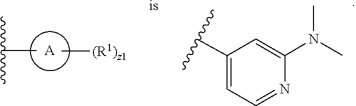

In embodiments,

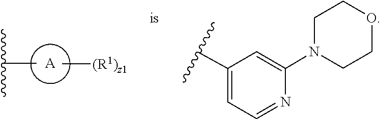

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is independently

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R² is independently halogen, —CF₃, —CHF₂, —CH₂F, —NH₂, substituted or unsubstituted C₁-C₈ alkyl, substituted or unsubstituted 2 to 8 membered heteroalkyl, substituted or unsubstituted C₃-C₅ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

In embodiments, R² is independently halogen. In embodiments, R² is independently

—F. In embodiments, R² is independently —Cl. In embodiments, R² is independently —Br. In embodiments, R² is independently —I. In embodiments, R² is independently —CCl₃. In embodiments, R² is independently —CBr₃. In embodiments, R² is independently —CF₃. In embodiments, R² is independently —CI₃. In embodiments, R² is independently —CH₂Cl. In embodiments, R² is independently —CH₂Br. In embodiments, R² is independently —CH₂F. In embodiments, R² is independently —CH₂I. In embodiments, R² is independently —CHCl₂. In embodiments, R² is independently —CHBr₂. In embodiments, R² is independently —CHF₂. In embodiments, R² is independently —CHI₂. In embodiments, R² is independently —CN. In embodiments, R² is independently —OH. In embodiments, R² is independently —NH₂. In embodiments, R² is independently —COOH. In embodiments, R² is independently —CONH₂. In embodiments, R² is independently —NO₂. In embodiments, R² is independently —SH. In embodiments, R² is independently —SO₃H. In embodiments, R² is independently —OSO₃H. In embodiments, R² is independently —SO₂NH₂. In embodiments, R² is independently —NHNH₂. In embodiments, R² is independently —ONH₂. In embodiments, R² is independently —NHC(O)NH₂. In embodiments, R² is independently —NHSO₂H. In embodiments, R² is independently —NHC(O)H. In embodiments, R² is independently —NHC(O)OH. In embodiments, R² is independently —NHOH. In embodiments, R² is independently —OCCl₃. In embodiments, R² is independently —OCBr₃. In embodiments, R² is independently —OCF₃. In embodiments, R² is independently —OCI₃. In embodiments, R² is independently —OCH₂Cl. In embodiments, R² is independently —OCH₂Br. In embodiments, R² is independently —OCH₂F. In embodiments, R² is independently —OCH₂I. In embodiments, R² is independently —OCHCl₂. In embodiments, R² is independently —OCHBr₂. In embodiments, R² is independently —OCHF₂. In embodiments, R² is independently —OCHI₂. In embodiments, R² is independently unsubstituted C₁-C₄ alkyl. In embodiments, R² is independently unsubstituted methyl. In embodiments, R² is independently unsubstituted ethyl. In embodiments, R² is independently unsubstituted propyl. In embodiments, R² is independently unsubstituted n-propyl. In embodiments, R² is independently unsubstituted isopropyl. In embodiments, R^{z} is independently unsubstituted butyl. In embodiments, R² is independently unsubstituted n-butyl. In embodiments, R² is independently unsubstituted isobutyl. In embodiments, R² is independently unsubstituted tert-butyl. In embodiments, R² is independently unsubstituted 2 to 4 membered heteroalkyl. In embodiments, R² is independently unsubstituted methoxy. In embodiments, R² is independently unsubstituted ethoxy. In embodiments, R² is independently unsubstituted propoxy. In embodiments, R² is independently unsubstituted n-propoxy. In embodiments, R² is independently unsubstituted isopropoxy. In embodiments, R² is independently unsubstituted butoxy. In embodiments, R² is independently unsubstituted n-butoxy. In embodiments, R² is independently unsubstituted isobutoxy. In embodiments, R² is independently unsubstituted tert-butoxy.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when RB is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B}substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R^{2A} is independently hydrogen. In embodiments, R^{2A} is independently unsubstituted C₁-C₄ alkyl. In embodiments, R^{2A} is independently unsubstituted methyl. In embodiments, R^{2A} is independently unsubstituted ethyl. In embodiments, R^{2A} is independently unsubstituted propyl. In embodiments, R^{2A} is independently unsubstituted n-propyl. In embodiments, R^{2A} is independently unsubstituted isopropyl. In embodiments, R^{2A} is independently unsubstituted butyl. In embodiments, R^{2A} is independently unsubstituted n-butyl. In embodiments, R^{2A} is independently unsubstituted isobutyl. In embodiments, R^{2A} is independently unsubstituted tert-butyl.

In embodiments, R^{2B} is independently hydrogen. In embodiments, R^{2B} is independently unsubstituted C₁-C₄ alkyl. In embodiments, R^{2B} is independently unsubstituted methyl. In embodiments, R^{2B} is independently unsubstituted ethyl. In embodiments, R^{2B} is independently unsubstituted propyl. In embodiments, R^{2B} is independently unsubstituted n-propyl. In embodiments, R^{2B} is independently unsubstituted isopropyl. In embodiments, R^{2B} is independently unsubstituted butyl. In embodiments, R^{2B} is independently unsubstituted n-butyl. In embodiments, R^{2B} is independently unsubstituted isobutyl. In embodiments, R^{2B} is independently unsubstituted tert-butyl.

In embodiments, R^{2C} is independently hydrogen. In embodiments, R^{2C} is independently unsubstituted C₁-C₄ alkyl. In embodiments, R^{2C} is independently unsubstituted methyl. In embodiments, R^{2C} is independently unsubstituted ethyl. In embodiments, R^{2C} is independently unsubstituted propyl. In embodiments, R^{2C} is independently unsubstituted n-propyl. In embodiments, R^{2C} is independently unsubstituted isopropyl. In embodiments, R^{2C} is independently unsubstituted butyl. In embodiments, R^{2C} is independently unsubstituted n-butyl. In embodiments, R^{2C} is independently unsubstituted isobutyl. In embodiments, R^{2C} is independently unsubstituted tert-butyl.

In embodiments, R^{2D} is independently hydrogen. In embodiments, R^{2D} is independently unsubstituted C₁-C₄ alkyl. In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted n-propyl. In embodiments, R^{2D} is independently unsubstituted isopropyl. In embodiments, R^{2D} is independently unsubstituted butyl. In embodiments, R^{2D} is independently unsubstituted n-butyl. In embodiments, R^{2D} is independently unsubstituted isobutyl. In embodiments, R^{2D} is independently unsubstituted tert-butyl.

In embodiments, z2 is 0. In embodiments, z2 is 1. In embodiments, z2 is 2. In embodiments, z2 is 3. In embodiments, z2 is 4.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen or unsubstituted C₁-C₆ alkyl. In embodiments, R³ is hydrogen. In embodiments, R³ is unsubstituted C₁-C₆ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl.

In embodiments, a substituted L⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L⁴ is substituted with a plurality of groups selected from substituent groups, size-limited substituent groups, and lower substituent groups; each substituent group, size-limited substituent group, and/or lower substituent group may optionally be different. In embodiments, when L⁴ is substituted, it is substituted with at least one substituent group. In embodiments, when L⁴ is substituted, it is substituted with at least one size-limited substituent group. In embodiments, when L⁴ is substituted, it is substituted with at least one lower substituent group.

In embodiments, L⁴ is a

bond, —C(O)—, —C(O)O—, —OC(O)—, —O—, —S—, —NH—, —C(O)NH—, —NHC(O)—, —NHC(O)O—, —OC(O)NH—, —NHC(O)NH—, —S(O)₂—, —NHS(O)₂—, —S(O)₂NH—, substituted or unsubstituted C₁-C₈ alkylene, substituted or unsubstituted 2 to 8 membered heteroalkylene, substituted or unsubstituted C₃-C₅ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L⁴ is a bond, —S—, —NH—, or substituted or unsubstituted 2 to 8 membered heteroalkylene.

In embodiments, L⁴ is a bond. In embodiments, L⁴ is —C(O)—. In embodiments, L⁴ is —C(O)O—. In embodiments, L⁴ is —OC(O)—. In embodiments, L⁴ is —O—. In embodiments, L⁴ is —S—. In embodiments, L⁴ is —NH—. In embodiments, L⁴ is —C(O)NH—. In embodiments, L⁴ is —NHC(O)—. In embodiments, L⁴ is —NHC(O)O—. In embodiments, L⁴ is —OC(O)NH—. In embodiments, L⁴ is —NHC(O)NH—. In embodiments, L⁴ is —S(O)₂—. In embodiments, L⁴ is —NHS(O)₂—. In embodiments, L⁴ is —S(O)₂NH—. In embodiments, L⁴ is substituted or unsubstituted 2 to 8 membered heteroalkylene. In embodiments, L⁴ is unsubstituted 2 to 8 membered heteroalkylene. In embodiments, 4 is.

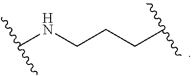 [SMILES: *CCCN*]

In embodiments, L⁴ is

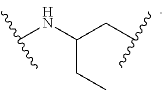 [SMILES: *CC(CC)N*]

In embodiments, L⁴ is

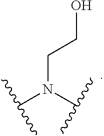 [SMILES: *N(*)CCO]

In embodiments, L⁴ is

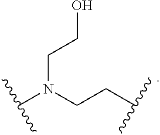 [SMILES: *CCN(*)CCO]

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁴ is hydrogen,

halogen, —CCl₃, —CBr₃, —CF₃, —CI₃, —CH₂Cl, —CH₂Br,
—CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is substituted or unsubstituted C₁-C₈ alkyl, substituted or unsubstituted 2 to 8 membered heteroalkyl, or substituted or unsubstituted C₃-C₈ cycloalkyl.

In embodiments, R⁴ is hydrogen. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CCl₃. In embodiments, R⁴ is —CBr₃. In embodiments, R⁴ is —CF₃. In embodiments, R⁴ is —CI₃. In embodiments, R⁴ is —CH₂C₁. In embodiments, R⁴ is —CH₂Br. In embodiments, R⁴ is —CH₂F. In embodiments, R⁴ is —CH₂J. In embodiments, R⁴ is —CHC1₂. In embodiments, R⁴ is —CHBr₂. In embodiments, R⁴ is —CHF₂. In embodiments, R⁴ is —CHI₂. In embodiments, R⁴ is —CN. In embodiments, R⁴ is —OH. In embodiments, R⁴ is —NH₂. In embodiments, R⁴ is —COOH. In embodiments, R⁴ is —CONH₂. In embodiments, R⁴ is —NO₂. In embodiments, R⁴ is —SH. In embodiments, R⁴ is —SO₃H. In embodiments, R⁴ is —OSO₃H. In embodiments, R⁴ is —SO₂NH₂. In embodiments, R⁴ is —NHNH₂. In embodiments, R⁴ is —ONH₂. In embodiments, R⁴ is —NHC(O)NH₂. In embodiments, R⁴ is —NHSO₂H. In embodiments, R⁴ is —NHC(O)H. In embodiments, R⁴ is —NHC(O)OH. In embodiments, R⁴ is —NHOH. In embodiments, R⁴ is —OCCl₃. In embodiments, R⁴ is —OCBr₃. In embodiments, R⁴ is —OCF₃. In embodiments, R⁴ is —OCl₃. In embodiments, R⁴ is —OCH₂Cl. In embodiments, R⁴ is —OCH₂Br. In embodiments, R⁴ is —OCH₂F. In embodiments, R⁴ is —OCH₂I. In embodiments, R⁴ is —OCHCl₂. In embodiments, R⁴ is —OCHBr₂. In embodiments, R⁴ is —OCHF₂. In embodiments, R⁴ is —OCHI₂. In embodiments, R⁴ is unsubstituted C₁-C₄ alkyl. In embodiments, R⁴ is unsubstituted methyl. In embodiments, R⁴ is unsubstituted ethyl. In embodiments, R⁴ is unsubstituted propyl. In embodiments, R⁴ is unsubstituted n-propyl. In embodiments, R⁴ is unsubstituted isopropyl. In embodiments, R⁴ is unsubstituted butyl. In embodiments, R⁴ is unsubstituted n-butyl. In embodiments, R⁴ is unsubstituted isobutyl. In embodiments, R⁴ is unsubstituted tert-butyl. In embodiments, R⁴ is unsubstituted 2 to 4 membered heteroalkyl. In embodiments, R⁴ is unsubstituted methoxy. In embodiments, R⁴ is unsubstituted ethoxy. In embodiments, R⁴ is unsubstituted propoxy. In embodiments, R⁴ is unsubstituted n-propoxy. In embodiments, R⁴ is unsubstituted isopropoxy. In embodiments, R⁴ is unsubstituted butoxy. In embodiments, R⁴ is unsubstituted n-butoxy. In embodiments, R⁴ is unsubstituted isobutoxy. In embodiments, R⁴ is unsubstituted tert-butoxy. In embodiments, R⁴ is unsubstituted C₃-C₈ cycloalkyl. In embodiments, R⁴ is unsubstituted unsubstituted cyclopropyl. In embodiments, R⁴ is unsubstituted cyclobutyl. In embodiments, R⁴ is unsubstituted cyclopentyl. In embodiments, R⁴ is unsubstituted cyclohexyl. In embodiments, R⁴ is unsubstituted cycloheptyl. In embodiments, R⁴ is unsubstituted cyclooctyl.

In embodiments, -L⁴-R⁴ is —SCH₃,

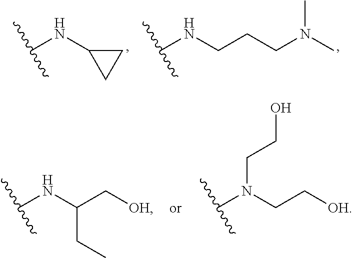

In embodiments, -L⁴-R⁴ is —SCH₃. In embodiments, -L⁴-R⁴ is

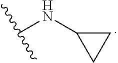 [SMILES: *NC1CC1]

In embodiments, -L⁴-R⁴ is

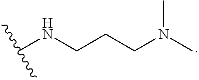 [SMILES: *NCCCN(C)C]

In embodiments, -L⁴-R⁴ is

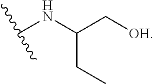 [SMILES: *NC(CC)CO]

In embodiments, -L⁴-R⁴ is

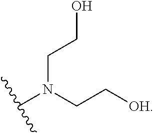 [SMILES: *N(CCO)CCO]

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B}substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R^{4A} is hydrogen. In embodiments, R^{4A} is unsubstituted C₁-C₄ alkyl. In embodiments, R^{4A} is unsubstituted methyl. In embodiments, R^{4A} is unsubstituted ethyl. In embodiments, R^{4A} is unsubstituted propyl. In embodiments, R^{4A} is unsubstituted n-propyl. In embodiments, R^{4A} is unsubstituted isopropyl. In embodiments, R^{4A} is unsubstituted butyl. In embodiments, R^{4A} is unsubstituted n-butyl. In embodiments, R^{4A} is unsubstituted isobutyl. In embodiments, R^{4A} is unsubstituted tert-butyl.

In embodiments, R^{4B} is hydrogen. In embodiments, R^{4B} is unsubstituted C₁-C₄ alkyl. In embodiments, R^{4B} is unsubstituted methyl. In embodiments, R^{4B} is unsubstituted ethyl. In embodiments, R^{4B} is unsubstituted propyl. In embodiments, R^{4B} is unsubstituted n-propyl. In embodiments, R^{4B} is unsubstituted isopropyl. In embodiments, R^{4B} is unsubstituted butyl. In embodiments, R^{4B} is unsubstituted n-butyl. In embodiments, R^{4B} is unsubstituted isobutyl. In embodiments, R^{4B} is unsubstituted tert-butyl.

In embodiments, R^{4C} is hydrogen. In embodiments, R^{4C} is unsubstituted C₁-C₄ alkyl. In embodiments, R^{4C} is unsubstituted methyl. In embodiments, R^{4C} is unsubstituted ethyl. In embodiments, R^{4C} is unsubstituted propyl. In embodiments, R^{4C} is unsubstituted n-propyl. In embodiments, R^{4C} is unsubstituted isopropyl. In embodiments, R^{4C} is unsubstituted butyl. In embodiments, R^{4C} is unsubstituted n-butyl. In embodiments, R^{4C} is unsubstituted isobutyl. In embodiments, R^{4C} is unsubstituted tert-butyl.

In embodiments, R^{4D} is hydrogen. In embodiments, R^{4D} is unsubstituted C₁-C₄ alkyl. In embodiments, R^{4D} is unsubstituted methyl. In embodiments, R^{4D} is unsubstituted ethyl. In embodiments, R^{4D} is unsubstituted propyl. In embodiments, R^{4D} is unsubstituted n-propyl. In embodiments, R^{4D} is unsubstituted isopropyl. In embodiments, R^{4D} is unsubstituted butyl. In embodiments, R^{4D} is unsubstituted n-butyl. In embodiments, R^{4D} is unsubstituted isobutyl. In embodiments, R^{4D} is unsubstituted tert-butyl.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, R¹⁰ is hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

In embodiments, R¹⁰ is hydrogen. In embodiments, R¹⁰ is unsubstituted C₁-C₄ alkyl. In embodiments, R¹⁰ is unsubstituted methyl. In embodiments, R¹⁰ is unsubstituted ethyl. In embodiments, R¹⁰ is unsubstituted propyl. In embodiments, R¹⁰ is unsubstituted n-propyl. In embodiments, R¹⁰ is unsubstituted isopropyl. In embodiments, R¹⁰ is unsubstituted butyl. In embodiments, R¹⁰ is unsubstituted n-butyl. In embodiments, R¹⁰ is unsubstituted isobutyl. In embodiments, R¹⁰ is unsubstituted tert-butyl.

In embodiments, a substituted R⁴⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴⁰ is substituted with a plurality of groups selected from substituent groups, size-limited substituent groups, and lower substituent groups; each substituent group, size-limited substituent group, and/or lower substituent group may optionally be different. In embodiments, when R⁴⁰ is substituted, it is substituted with at least one substituent group. In embodiments, when R⁴⁰ is substituted, it is substituted with at least one size-limited substituent group. In embodiments, when R⁴⁰ is substituted, it is substituted with at least one lower substituent group.

In embodiments, R⁴⁰ is hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

In embodiments, R⁴⁰ is hydrogen. In embodiments, R⁴⁰ is unsubstituted C₁-C₄ alkyl. In embodiments, R⁴⁰ is unsubstituted methyl. In embodiments, R⁴⁰ is unsubstituted ethyl. In embodiments, R⁴⁰ is unsubstituted propyl. In embodiments, R⁴⁰ is unsubstituted n-propyl. In embodiments, R⁴⁰ is unsubstituted isopropyl. In embodiments, R⁴⁰ is unsubstituted butyl. In embodiments, R⁴⁰ is unsubstituted n-butyl. In embodiments, R⁴⁰ is unsubstituted isobutyl. In embodiments, R⁴⁰ is unsubstituted tert-butyl.

In embodiments, a substituted R^{40A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{40A} is substituted with a plurality of groups selected from substituent groups, size-limited substituent groups, and lower substituent groups; each substituent group, size-limited substituent group, and/or lower substituent group may optionally be different. In embodiments, when R^{40A} is substituted, it is substituted with at least one substituent group. In embodiments, when R^{40A} is substituted, it is substituted with at least one size-limited substituent group. In embodiments, when R^{40A} is substituted, it is substituted with at least one lower substituent group.

In embodiments, R^{40A} is

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

In embodiments, R^{40A} is hydrogen. In embodiments, R⁴⁰A is unsubstituted C₁-C₄ alkyl. In embodiments, R^{40A} is unsubstituted methyl. In embodiments, R⁴⁰A is unsubstituted ethyl. In embodiments, R⁴⁰A is unsubstituted propyl. In embodiments, R⁴⁰A is unsubstituted n-propyl. In embodiments, R^{40A} is unsubstituted isopropyl. In embodiments, R⁴⁰A is unsubstituted butyl. In embodiments, R^{40A} is unsubstituted n-butyl. In embodiments, R^{40A} is unsubstituted isobutyl. In embodiments, R^{4A} is unsubstituted tert-butyl.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A}., R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B. 1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B. 1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R, R^{WW.2}, and R^{WW3}, respectively, as explained in the definitions section above in the description of “first substituent group(s)”, wherein R^{WW.1}, R^{WW.2}, and R^{WW.3} correspond to R^{1A.1}, R^{1A.2} and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}R², and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C}. substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D}.2 substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to RID, R^{1D.1}, R^{1D.2}, and R^{1D.3}, respectively.

In embodiments, when R² is substituted, R² is substituted with one or more first substituent groups denoted by R²1 as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, RWWi, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B3}. as explained in the definitions section above in the description of “first substituent group(s)”. In the above embodiments, R^{2B}, R^{2B.1}, R^{2B.2}, and R^{2B3}. have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2} and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R³¹ as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R⁴A² substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R″—W, R^{WW.2}, and R′—³ correspond to R^{4A.1}, R^{4A.2} and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4D}, R^{4D.1}, R^{4D.2}, and R^{4D.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R¹⁰¹, R^{10.2}, and R^{10.3} have values corresponding to the values of R^{WW}, R^{WW.1}, R^{WW.2}, and R^{WW.3}, respectively, as explained in the definitions section above in the description of “first substituent group(s)”, wherein R^{W}, R^{WW.1}, R^{WW.2}, and R^{WW.3} correspond to R¹⁰, R^{10.1}, R^{10.2}, and R^{10.3}, respectively.

In embodiments, when R⁴⁰ is substituted, R⁴⁰ is substituted with one or more first substituent groups denoted by R^{40.1} as explained in the definitions section above in the description of “first substituent group(s)”. In embodiments, when an R^{40.1} substituent group is substituted, the R^{40.1} substituent group is substituted with one or more second substituent groups denoted by R^{40.2} as explained in the definitions section above in the description of “first substituent group(s)”. In embodiments, when an R^{40.2} substituent group is substituted, the R^{40.2} substituent group is substituted with one or more third substituent groups denoted by R^{40.3} as explained in the definitions section above in the description of “first substituent group(s)”. In the above embodiments, R⁴⁰, R⁴⁰¹, R^{40.2}, and R^{40.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴⁰, R⁴⁰¹, R^{40.2}, and R⁴⁰³, respectively.

In embodiments, when R⁴⁰A is substituted, R⁴⁰A is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{40A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when L⁴ is substituted, L⁴ is substituted with one or more first substituent groups denoted by R^{L4.1} as explained in the definitions section above in the description of “first substituent group(s)”. In embodiments, when an R^{L4.1} substituent group is substituted, the R^{L4.1} substituent group is substituted with one or more second substituent groups denoted by R^{L4.2} as explained in the definitions section above in the description of “first substituent group(s)”. In embodiments, when an R^{L4.2} substituent group is substituted, the R^{L4.2} substituent group is substituted with one or more third substituent groups denoted by R^{L4.3} as explained in the definitions section above in the description of “first substituent group(s)”. In the above embodiments, L⁴, R^{L4.1}, R^{L4.2}, and R^{L4.3} have values corresponding to the values of L^{WW}, R^{LWW.1}, R^{LWW.2}, and R^{LWW.3}, respectively, as explained in the definitions section above in the description of “first substituent group(s)”, wherein L″, R^{LWW.1}, R^{LWW.2}, and R^{LWW.3} are L⁴, R^{L4.1}, R^{L4.2}, and R^{L4.3}, respectively.

In embodiments, the compound has the formula:

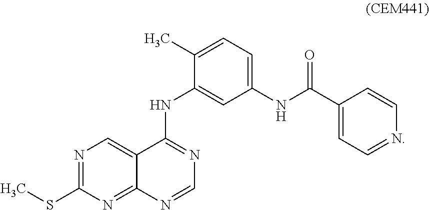 [SMILES: CSc1ncc2c(Nc3cc(NC(=O)c4ccncc4)ccc3C)ncnc2n1]

In embodiments, the compound has the formula:

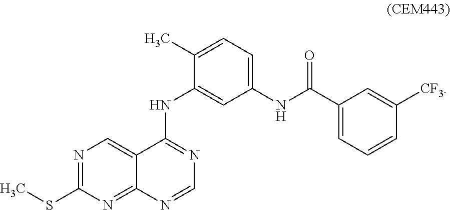 [SMILES: CSc1ncc2c(Nc3cc(NC(=O)c4cccc(C(F)(F)F)c4)ccc3C)ncnc2n1]

In embodiments, the compound has the formula:

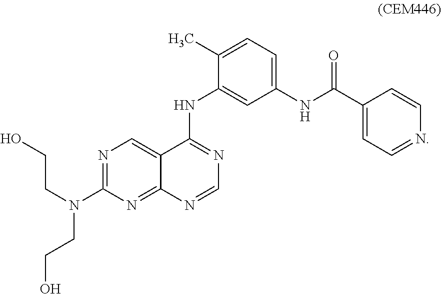 [SMILES: Cc1ccc(NC(=O)c2ccncc2)cc1Nc1ncnc2nc(N(CCO)CCO)ncc12]

In embodiments, the compound has the formula:

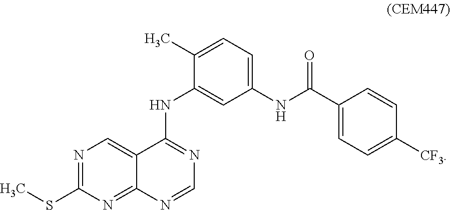 [SMILES: CSc1ncc2c(Nc3cc(NC(=O)c4ccc(C(F)(F)F)cc4)ccc3C)ncnc2n1]

In embodiments, the compound has the formula:

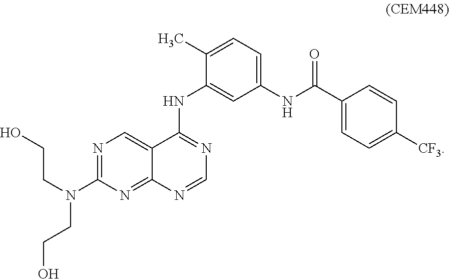 [SMILES: Cc1ccc(NC(=O)c2ccc(C(F)(F)F)cc2)cc1Nc1ncnc2nc(N(CCO)CCO)ncc12]

In embodiments, the compound has the formula:

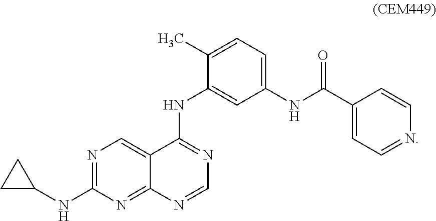 [SMILES: Cc1ccc(NC(=O)c2ccncc2)cc1Nc1ncnc2nc(NC3CC3)ncc12]

In embodiments, the compound has the formula:

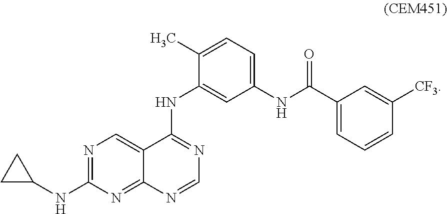 [SMILES: Cc1ccc(NC(=O)c2cccc(C(F)(F)F)c2)cc1Nc1ncnc2nc(NC3CC3)ncc12]

In the embodiments, the compound has the formula:

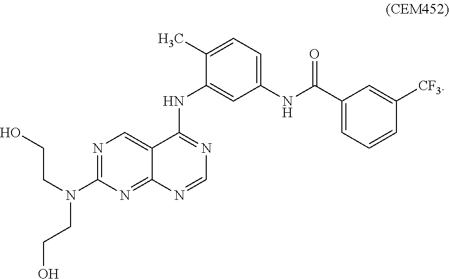 [SMILES: Cc1ccc(NC(=O)c2cccc(C(F)(F)F)c2)cc1Nc1ncnc2nc(N(CCO)CCO)ncc12]

In embodiments, the compound has the formula:

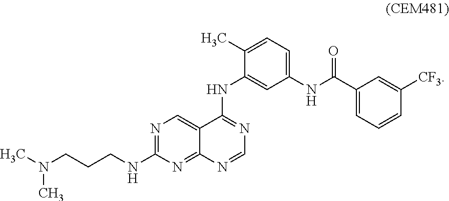 [SMILES: Cc1ccc(NC(=O)c2cccc(C(F)(F)F)c2)cc1Nc1ncnc2nc(NCCCN(C)C)ncc12]

In embodiments, the compound has the formula:

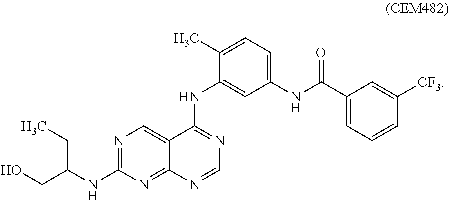 [SMILES: CCC(CO)Nc1ncc2c(Nc3cc(NC(=O)c4cccc(C(F)(F)F)c4)ccc3C)ncnc2n1]

In embodiments, the compound has the formula:

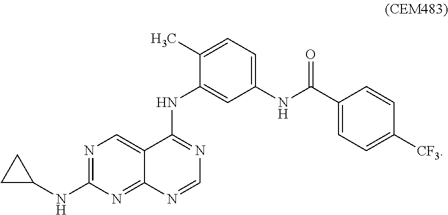 [SMILES: Cc1ccc(NC(=O)c2ccc(C(F)(F)F)cc2)cc1Nc1ncnc2nc(NC3CC3)ncc12]

In embodiments, the compound has the formula:

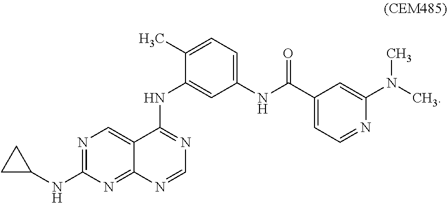 [SMILES: Cc1ccc(NC(=O)c2ccnc(N(C)C)c2)cc1Nc1ncnc2nc(NC3CC3)ncc12]

In embodiments, the compound has the formula:

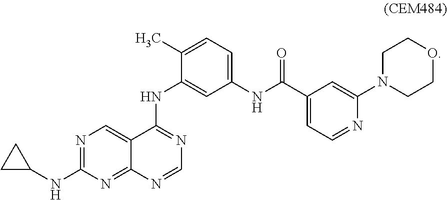 [SMILES: Cc1ccc(NC(=O)c2ccnc(N3CCOCC3)c2)cc1Nc1ncnc2nc(NC3CC3)ncc12]

In embodiments, the compound contacts an amino acid corresponding to Lys53, Glu71, Met109, Asp168, or Phe169 of human p38α protein. In embodiments, the compound contacts an amino acid corresponding to Lys53 of human p38α protein. In embodiments, the compound contacts an amino acid corresponding to Glu71 of human p38α protein. In embodiments, the compound contacts an amino acid corresponding to Met109 of human p38α protein. In embodiments, the compound contacts an amino acid corresponding to Asp168 of human p38α protein. In embodiments, the compound contacts an amino acid corresponding to Phe169 of human p38α protein.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound. In embodiments, the compound is a compound of formula (I), (II), (III), or (IV), including embodiments thereof.

In embodiments, the pharmaceutical composition further includes an epigenetic inhibitor. In embodiments, the epigenetic inhibitor is a histone deacetylase (HDAC) inhibitor. In embodiments, the epigenetic inhibitor is an FDA-approved epigenetic drug. In embodiments, the FDA-approved epigenetic drug is an HDAC inhibitor drug. In embodiments, the HDAC inhibitor (e.g., HDAC inhibitor drug) is vorinostat (SAHA). In embodiments, the HDAC inhibitor (e.g., HDAC inhibitor drug) is romidepsin. In embodiments, the epigenetic inhibitor is a DNA methyltransferase (DNMT) inhibitor. In embodiments, the epigenetic inhibitor is an EZH2 inhibitor.

In embodiments, the pharmaceutical composition includes a synergistically effective amount of the compound. In embodiments, the pharmaceutical composition includes a synergistically effective amount of the HDAC inhibitor.

### IV. Methods of Use

In an aspect is provided a method of treating cancer in a subject in need thereof, the method including administering to the subject a therapeutically effective amount of a compound described herein, or a pharmaceutically acceptable salt thereof.

In embodiments, the cancer is lymphoma, leukemia, multiple myeloma, prostate cancer, breast cancer, bladder cancer, liver cancer, lung cancer, ovarian cancer, glioblastoma, pancreatic cancer, renal cancer, head and neck cancer, colon cancer, or melanoma. In embodiments, the cancer is lymphoma. In embodiments, the cancer is cutaneous T-cell lymphoma. In embodiments, the cancer is leukemia. In embodiments, the cancer is multiple myeloma. In embodiments, the cancer is prostate cancer. In embodiments, the cancer is breast cancer. In embodiments, the cancer is bladder cancer. In embodiments, the cancer is liver cancer. In embodiments, the cancer is lung cancer. In embodiments, the cancer is ovarian cancer. In embodiments, the cancer is glioblastoma. In embodiments, the cancer is pancreatic cancer. In embodiments, the cancer is renal cancer. In embodiments, the cancer is head and neck cancer. In embodiments, the cancer is colon cancer. In embodiments, the cancer is melanoma.

In embodiments, the method further includes administering a therapeutically effective amount of an epigenetic inhibitor. In embodiments, the method further includes administering in combination a therapeutically effective amount of an epigenetic inhibitor. In embodiments, the epigenetic inhibitor is a histone deacetylase (HDAC) inhibitor. In embodiments, the epigenetic inhibitor is an FDA-approved epigenetic drug. In embodiments, the FDA-approved epigenetic drug is an HDAC inhibitor drug. In embodiments, the HDAC inhibitor (e.g., HDAC inhibitor drug) is vorinostat (SAHA). In embodiments, the HDAC inhibitor (e.g., HDAC inhibitor drug) is romidepsin. In embodiments, the epigenetic inhibitor is a DNA methyltransferase (DNMT) inhibitor. In embodiments, the epigenetic inhibitor is an EZH2 inhibitor.

In an aspect is provided a method of treating a p38 MAPK-associated disease in a subject in need thereof, the method including administering to the subject a therapeutically effective amount of a compound described herein, or a pharmaceutically acceptable salt thereof.

In embodiments, the p38 MAPK-associated disease is cancer (e.g., lymphoma, leukemia, multiple myeloma, prostate cancer, breast cancer, bladder cancer, liver cancer, lung cancer, ovarian cancer, or glioblastoma). In embodiments, the p38 MAPK-associated disease is an autoimmune disease. In embodiments, the p38 MAPK-associated disease is an inflammatory disease. In embodiments, the p38 MAPK-associated disease is chronic obstructive pulmonary disease.

In an aspect is provided a method of modulating the level of activity of a p38 MAPK (e.g., p38α or p38β) protein in a cell, the method including contacting the cell with an effective amount of a compound described herein, or a pharmaceutically acceptable salt thereof. In embodiments, the p38 MAPK (e.g., p38α or p38β) protein is a human p38 MAPK (e.g., human p38α or human p38β) protein.

In embodiments, the modulating is reducing the activity of the p38 MAPK (e.g., p38α or p38β) protein. In embodiments, the level of activity of the p38 MAPK (e.g., p38α or p38β) protein is reduced by about 1.5-, 2—, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the p38 MAPK (e.g., p38α or p38β) protein is reduced by at least 1.5-, 2—, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound).

### V. EMBODIMENTS

Embodiment P1. A compound, or a pharmaceutically acceptable salt thereof, having the formula:

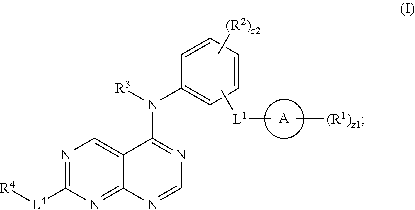

wherein
Ring A is cycloalkyl, heterocycloalkyl, aryl, or heteroaryl;
L¹ is —NR¹⁰C(O)— or —C(O)NR¹⁰—
L⁴ is a bond, —C(O)—, —C(O)O—, —OC(O)—, —O—, —S—, —NR⁴⁰—, —C(O)NR⁴⁰—,
—NR⁴⁰C(O)—, —NR⁴⁰C(O)O—, —OC(O)NR⁴⁰—, —NR⁴⁰C(O)NR^{40A}—, —(O)₂—, —NR⁴⁰S(O)₂—,
—S(O)₂NR⁴⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹,
—OCHX¹², —CN, —SOn₁R^{1D}, —SOᵥ₁NR^{1A}R^{1B}, —NRicNR^{1A}R^{1B}, —ONR^{1A}R^{1B}, —NR^{1C}C(O)NR^{1A}R^{1B},
—N(O)ₘ₁, —NR^{1A}R^{1B}—C(O)R^{1C}, —C(O)OR^{1C}, —OC(O)R^{1C}, —OC(O)OR^{1C}, —C(O)NR^{1A}R^{1B},
—OC(O)NR^{1A}R^{1B}—OR^{1D}, —SR^{1D}, —NR^{1A}SO₂R^{1D}, —NR^{1A}C(O)R^{1C}, —NR^{1A}C(O)OR^{1C}, —NR^{1A}OR^{1C},
—SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²², —CH₂X², —OCX²₃, —OCH₂X²,
—OCHX²², —CN, —SOn₂R^{2D}, —SOᵥ₂NR^{2A}R^{2B}, —NR²cNR^{2A}R^{2B}, —ONR^{2A}R^{2B}, —NR^{2C}C(O)NR^{2A}R^{2B},
—N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B},
—OC(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C},
—SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —Cl₃, —CH₂Cl, —CH₂Br, —CH₂F, —CH₂I, —CHC1₂,
—CHBr₂, —CHF₂, —CHI₂, —CN, —OH, —NH₂, —COOH, —CONH₂, —OCCl₃, —OCBr₃, —OCF₃, —OCI₃,
—OCH₂Cl, —OCH₂Br, —OCH₂F, —OCH₂I, —OCHC1₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂,
—CN, —SOn₄R⁴D, —SO₄NR^{4A}R^{4B}, NR⁴cNR^{4A}R^{4B}—ONR^{4A}R^{4B}, NHC(O)NR^{4A}R^{4B}, N(O)ₘ₄,
—NR^{4A}R^{4B}—C(O)R^{4C}, —C(O)OR^{4C}, —C(O)NR^{4A}R^{4B}—OR^{4D}, —SR^{4D}, —NR⁴ASO₂R⁴D, —NR⁴AC(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰, R⁴⁰, and R^{40A} are independently hydrogen, —CCl₃, —CBr₃, —CF₃, —CI₃, —CH₂Cl,
—CH₂Br, —CH₂F, —CH₂I, —CHCl₂, —CHBr₂, —CHF₂, —CHI₂, —CN, —OH, —NH₂, —COOH, —CONH₂,
—OCCl₃, —OCBr₃, —OCF₃, —OCI₃, —OCH₂Cl, —OCH₂Br, —OCH₂F, —OCH₂I, —OCHCl₂, —OCHBr₂,
—OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F,
—CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{IB} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
z1 is an integer from 0 to 7;
z2 is an integer from 0 to 4;
X¹, X², and X⁴ are independently —Cl, —Br, —I, or —F;
n1, n2, and n4 are independently an integer from 0 to 4; and
m1, m2, m4, v1, v2, and v4 are independently 1 or 2.

Embodiment P2. The compound of embodiment P1, having the formula:

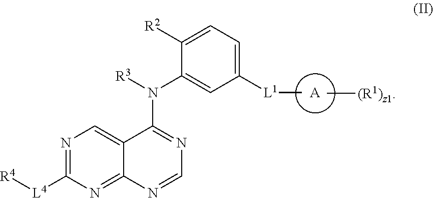

Embodiment P3. The compound of one of embodiments P1 to P2, having the formula:

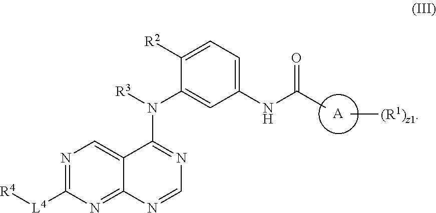

Embodiment P4. The compound of one of embodiments P1 to P2, having the formula:

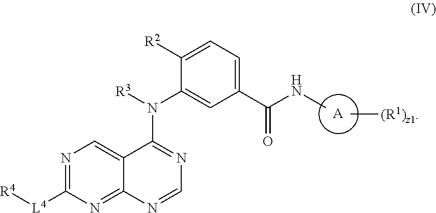

Embodiment P5. The compound of one of embodiments P1 to P4, wherein Ring A is phenyl or 5 to 6 membered heteroaryl.

Embodiment P6. The compound of one of embodiments P1 to P4, wherein Ring A is phenyl.

Embodiment P7. The compound of one of embodiments P1 to P4, wherein Ring A is pyridinyl.

Embodiment P8. The compound of one of embodiments P1 to P7, wherein R¹ is independently

halogen, —CCl₃, —CBr₃, —CF₃, —CI₃, —CH₂Cl, —CH₂Br, —CH₂F, —CH₂I, —CHC1₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHC1₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P9. The compound of one of embodiments P1 to P7, wherein R¹ is independently halogen, —CF₃, —CHF₂, —CH₂F, —NH₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P10. The compound of one of embodiments P1 to P7, wherein R¹ is independently —CF₃, —N(CH₃)₂, or unsubstituted morpholinyl.

Embodiment P11. The compound of one of embodiments P1 to P10, wherein z1 is 1.

Embodiment P12. The compound of one of embodiments P1 to P7, wherein z1 is 0.

Embodiment P13. The compound of one of embodiments P1 to P7, wherein

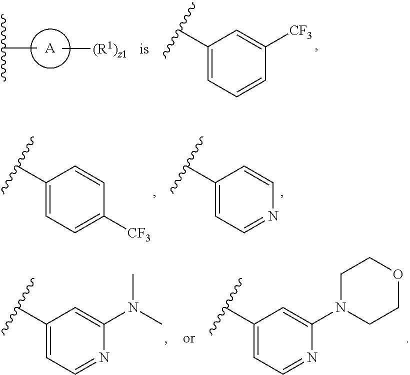

Embodiment P14. The compound of one of embodiments P1 to P12, wherein R² is independently

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P15. The compound of one of embodiments P1 to P12, wherein R² is independently halogen, —CF₃, —CHF₂, —CH₂F, —NH₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P16. The compound of one of embodiments P1 to P12, wherein R² is independently unsubstituted C₁-C₄ alkyl.

Embodiment P17. The compound of one of embodiments P1 to P12, wherein R² is independently unsubstituted methyl.

Embodiment P18. The compound of one of embodiments P1 to P17, wherein R³ is hydrogen or unsubstituted C₁-C₆ alkyl.

Embodiment P19. The compound of one of embodiments P1 to P17, wherein R³ is hydrogen.

Embodiment P20. The compound of one of embodiments P1 to P19, wherein L⁴ is a bond, —C(O)—, —C(O)O—, —OC(O)—, —O—, —S—, —NH—, —C(O)NH—, —NHC(O)—, —NHC(O)O—, —OC(O)NH—, —NHC(O)NH—, —S(O)₂—, —NHS(O)₂—, —S(O)₂NH—, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

Embodiment P21. The compound of one of embodiments P1 to P19, wherein L⁴ is a bond, —S—, —NH—, or substituted or unsubstituted 2 to 8 membered heteroalkylene.

Embodiment P22. The compound of one of embodiments P1 to P21, wherein R⁴ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P2₃. The compound of one of embodiments P1 to P21, wherein R⁴ is substituted or unsubstituted C₁-C₈ alkyl, substituted or unsubstituted 2 to 8 membered heteroalkyl, or substituted or unsubstituted C₃-C₈ cycloalkyl.

Embodiment P24. The compound of one of embodiments P1 to P19, wherein -L⁴-R⁴ is —SCH₃,

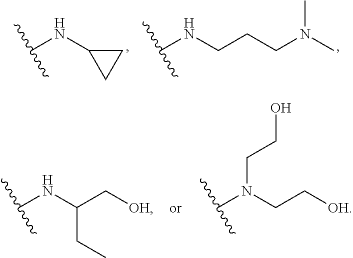

Embodiment P25. The compound of embodiment P1, having the formula:

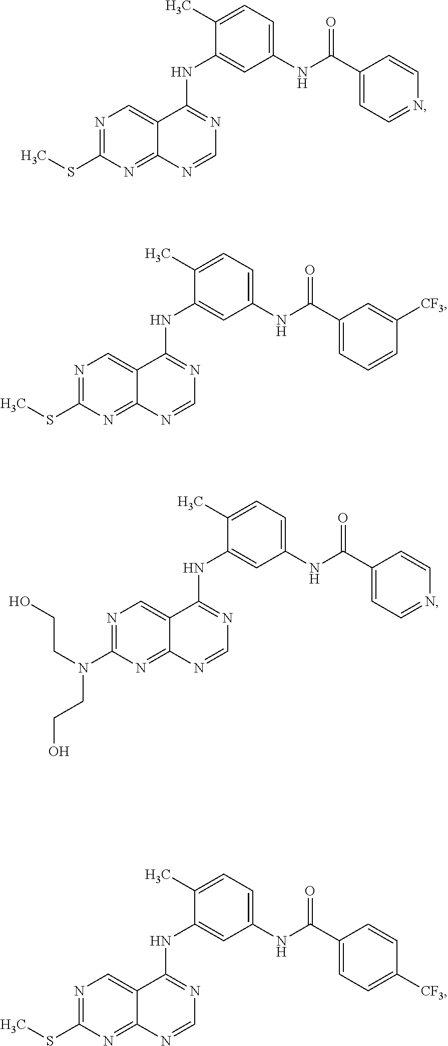 [SMILES: CSc1ncc2c(Nc3cc(NC(=O)c4ccncc4)ccc3C)ncnc2n1 ; CSc1ncc2c(Nc3cc(NC(=O)c4cccc(C(F)(F)F)c4)ccc3C)ncnc2n1 ; Cc1ccc(NC(=O)c2ccncc2)cc1Nc1ncnc2nc(N(CCO)CCO)ncc12 ; CSc1ncc2c(Nc3cc(NC(=O)c4ccc(C(F)(F)F)cc4)ccc3C)ncnc2n1]

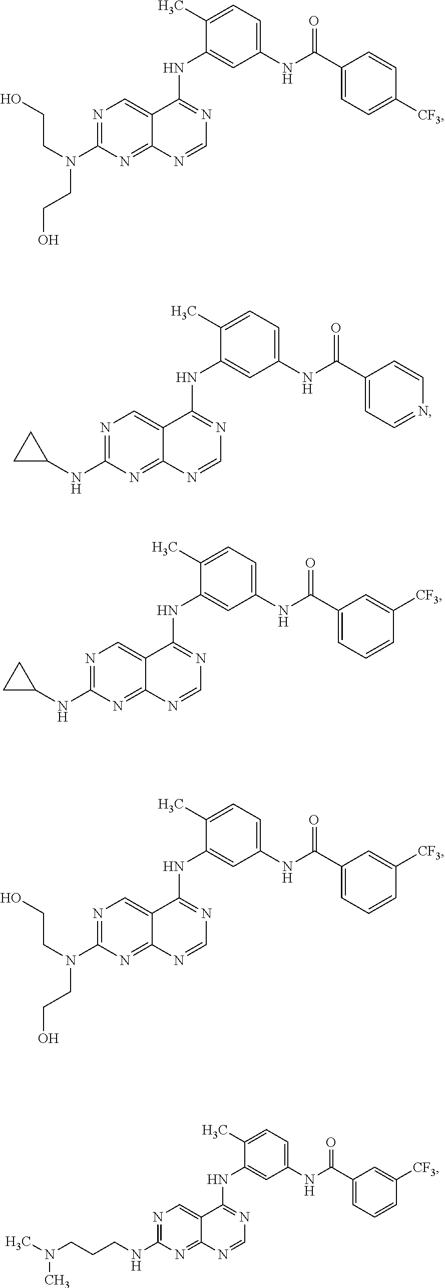 [SMILES: Cc1ccc(NC(=O)c2ccc(C(F)(F)F)cc2)cc1Nc1ncnc2nc(N(CCO)CCO)ncc12 ; Cc1ccc(NC(=O)c2ccncc2)cc1Nc1ncnc2nc(NC3CC3)ncc12 ; Cc1ccc(NC(=O)c2cccc(C(F)(F)F)c2)cc1Nc1ncnc2nc(NC3CC3)ncc12 ; Cc1ccc(NC(=O)c2cccc(C(F)(F)F)c2)cc1Nc1ncnc2nc(N(CCO)CCO)ncc12 ; Cc1ccc(NC(=O)c2cccc(C(F)(F)F)c2)cc1Nc1ncnc2nc(NCCCN(C)C)ncc12]

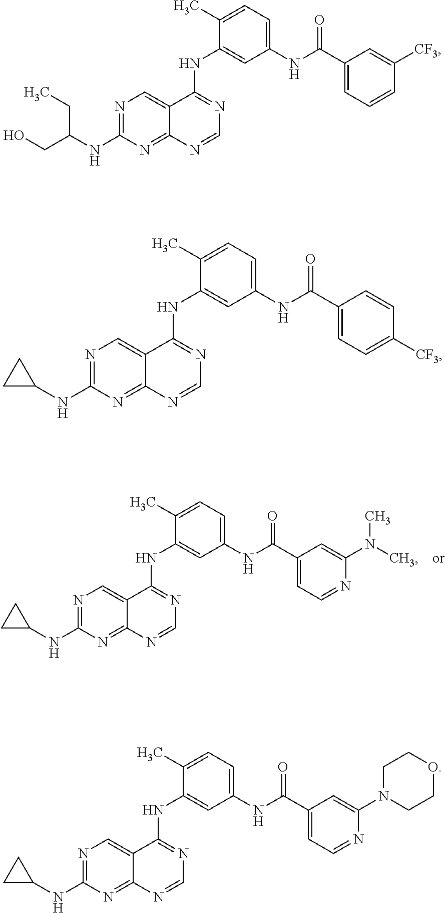 [SMILES: CCC(CO)Nc1ncc2c(Nc3cc(NC(=O)c4cccc(C(F)(F)F)c4)ccc3C)ncnc2n1 ; Cc1ccc(NC(=O)c2ccc(C(F)(F)F)cc2)cc1Nc1ncnc2nc(NC3CC3)ncc12 ; Cc1ccc(NC(=O)c2ccnc(N(C)C)c2)cc1Nc1ncnc2nc(NC3CC3)ncc12 ; Cc1ccc(NC(=O)c2ccnc(N3CCOCC3)c2)cc1Nc1ncnc2nc(NC3CC3)ncc12]

Embodiment P26. A pharmaceutical composition comprising a compound of one of embodiments P1 to P25 or a pharmaceutically acceptable salt thereof, and a pharmaceutically acceptable excipient.

Embodiment P27. The pharmaceutical composition of embodiment P26, further comprising an epigenetic inhibitor.

Embodiment P28. The pharmaceutical composition of embodiment P27, wherein the epigenetic inhibitor is an HDAC inhibitor.

Embodiment P29. The pharmaceutical composition of embodiment P28, wherein the HDAC inhibitor is SAHA.

Embodiment P30. The pharmaceutical composition of embodiment P28, wherein the HDAC inhibitor is romidepsin.

Embodiment P31. A method of treating cancer in a subject in need thereof, said method comprising administering to the subject a therapeutically effective amount of a compound of one of embodiments P1 to P25, or a pharmaceutically acceptable salt thereof.

Embodiment P32. The method of embodiment P31, wherein said cancer is lymphoma, leukemia, multiple myeloma, prostate cancer, breast cancer, bladder cancer, liver cancer, lung cancer, ovarian cancer, glioblastoma, pancreatic cancer, renal cancer, head and neck cancer, colon cancer, or melanoma.

Embodiment P33. The method of embodiment P31, wherein said cancer is cutaneous T-cell lymphoma.

Embodiment P34. The method of one of embodiments P31 to P33, further comprising administering a therapeutically effective amount of an HDAC inhibitor.

Embodiment P35. The method of embodiment P34, wherein the HDAC inhibitor is SAHA.

Embodiment P36. The method of embodiment P34, wherein the HDAC inhibitor is romidepsin.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

Quinazoline derivatives were initially tested with various cancer types of cell lines using a cell-based assay. Several small molecule compounds reduced viabilities of these cell lines at 1 M or 10 M concentration. CEM451 potently inhibited both p38 MAPK alpha and beta catalytic activities with IC₅₀=6.2 nM and 1₃ nM, respectively, suggesting that the compound directly targets these kinases. The compound broadly showed antitumor activities against hematopoietic cell lines. In particular, CEM451 revealed IC₅₀=0.54 M against K562 CML cells.

Currently, SAHA (Vorinostat) and Romidepsin are used for treatment of cutaneous T-cell lymphoma (CTCL). SAHA is undergoing clinical trial studies in multiple myeloma (MM) treatment. CEM451 displayed synergistic effects in combination with SAHA in Hut78 CTCL and RPMI8226 MM cells. These results suggest that inhibition of p38 alpha could synergistically enhance efficacies of HDAC inhibitors in human tumor treatment.

### Example 2: Experimental Methods

The synthesis of the amino-substituted pyrimidinopyrimidines started from nitriles 6a-6e which upon reaction with anilines 11a-11c and 17a-17b afforded the desired compounds 7a-7m through standard Dimroth rearrangement conditions (Scheme 1). Regarding nitriles 6a-6c, thiourea 1 was reacted with dimethyl sulphate to afford compound 2 according to Shildneck P. R. et al. (Shildneck P. R, Windus W.; Organic Syntheses 411, 1943) (Scheme 1). Reaction of 2 with 2-(ethoxymethylene)malononitrile afforded aminopyrimidine 3 in 89% yield (Katiyar S. B., Kumar A., Chauhan P. M. S.; Synth. Commun. 36, 2963.2973, 2006). Thiomethyl compound 3 was then oxidized to the methylsulfonyl compound 4 under treatment with m-chloroperbenzoic acid (Katiyar S. B., Kumar A., Chauhan P. M. S.; Synth. Commun. 36, 2963.2973, 2006), which upon reaction with the suitable amines afforded cyanopyrimidines 5a-5b. Finally, treatment of compounds 5a-5b with DMF-DMA afforded nitriles 6a-6b. For the synthesis of 6c, compound 3 was used as starting material following the same procedure (Branko, S., Uros, U., Miha, T.; Heterocycles, 23, 2819.2828, 1985).

The synthetic procedure of anilines 11a-11c is depicted in Scheme 2. For the preparation of nitroaniline 9, we followed the previously reported approach by Kock, M. et al. (Kock, M., Jones, P. G., Linde, T.; Org. Lett., 19, 6296.6299, 2017). Subsequently, aniline 9 was reacted with the suitable carboxylic acid to provide amides 10a-10c, which upon reduction by SnCl₂ afforded the desired anilines 11a-11c.

Anilines 17a-17b were prepared according to Scheme 3. Commercially available isonicotinic acid 12 was first oxidized to the corresponding N-oxide 1₃ (Bailey, A. J., Griffith, W. P., Parkin, B. C.; J. Chem. Soc. Dalton Transactions, 1833.1838, 1995) and then reacted with POCl₃, to provide the chloronicotinic acid 14 (Anderson, W. K., Dean, D. C., Endo, T.; J. Med. Chem. 33, 1667.1675, 1990). The next step of the synthetic procedure involves the synthesis of amide 15 through reaction with 4-methyl-3-nitroaniline to afford chloride 15 (EP1165566, 2003, B1). Reaction of compound 15 with morpholine or dimethylamine resulted in compounds 16α (W02003/90912, 2003, A1) and 16b, which upon hydrogenation with Pd/C afforded the desired anilines 17α (U.S. Pat. No. 6,593,333, 2003, B1) and 17b respectively.

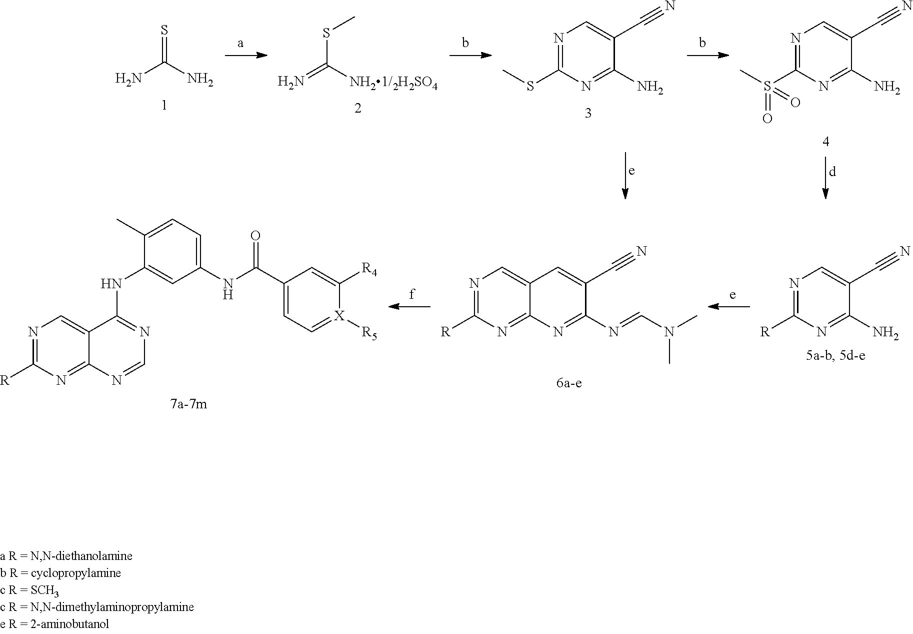

Scheme 1: Reactions and conditions: a) Dimethyl sulfate, H₂O, 80° C.; b) 2-(ethoxymethylene)malononitrile, triethylamine, ethanol abs., rt; c) mCPBA, CH₂Cl₂, r.t.; d) suitable amine, THF/methanol, r.t.; e) DMF-DMA, toluene, reflux; f) suitable aniline, CH₃CO₂H, reflux.

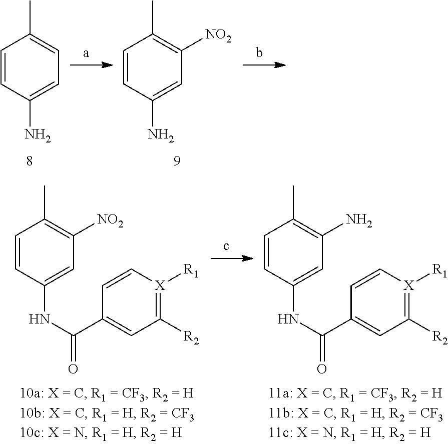

Scheme 2: Reactions and conditions: a) c. HNO₃, c. H₂SO₄, 0° C., rt; b) suitable aryl chloride, triethylamine, THF, rt; or suitable acid, EDCI-HCl, DMF, rt; c) SnCl₂—H₂O, acetone, rt.

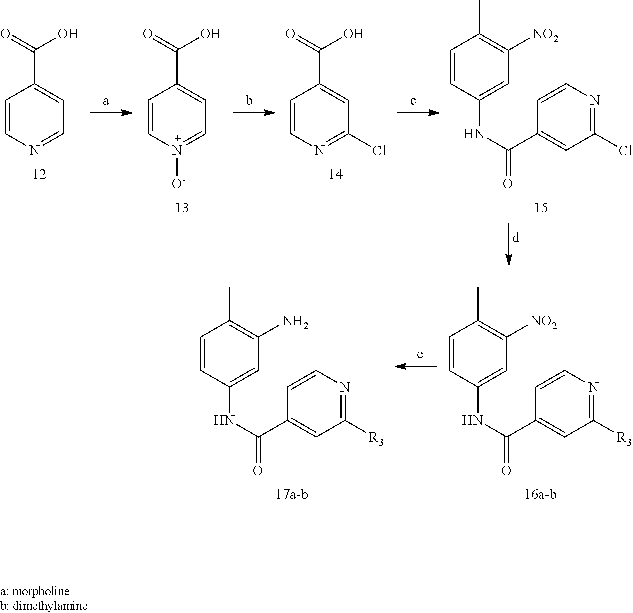

Scheme 3: Reactions and conditions: a) H₂O₂, acetic acid, 70.80° C.; b) POCl₃, reflux; c) i) SOCl₂, 80° C.; ii) 4-methyl-3-nitroaniline, Et₃N, THF, 0° C., rt; d) suitable amine, reflux; e) H₂, Pd/C, ethanol abs, 1 atm, rt.

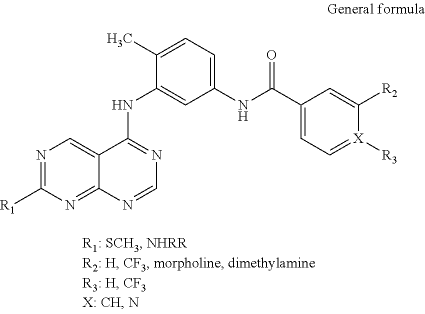

| Compound | X | R₁ | R₂ | R₃ |
|---|---|---|---|---|
| 7a (CEM452) | C | N,N-diethanolamine | CF₃ | H |
| 7b (CEM448) | C | N,N-diethanolamine | H | CF₃ |
| 7c (CEM446) | N | N,N-diethanolamine | H | n/a |
| 7d (CEM451) | C | cyclopropylamine | CF₃ | H |
| 7e (CEM483) | C | cyclopropylamine | H | CF₃ |
| 7f (CEM449) | N | cyclopropylamine | H | n/a |
| 7g (CEM484) | N | cyclopropylamine | morpholine | n/a |
| 7h (CEM485) | N | cyclopropylamine | dimethylamine | n/a |
| 7i (CEM443) | C | SCH₃ | CF₃ | H |
| 7j (CEM447) | C | SCH₃ | H | CF₃ |
| 7k (CEM441) | N | SCH₃ | H | n/a |
| 7l (CEM481) | C | N,N-dimethylaminopropylamine | CF₃ | H |
| 7m (CEM482) | C | 2-aminoboutanol | CF₃ | H |

### Experimental Procedures

All commercially available reagents and solvents were purchased from Alfa Aesar and used without any further purification. Melting points were determined on a Buichi apparatus and were uncorrected. All NMR spectra were recorded on 400 or 600 MHz Bruker spectrometers respectively Avance™ DRX and III instruments (Bruker BioSpin GmbH -Rheinstetten, Germany). ¹H NMR (400 and 600 MHz) and ¹³C NMR (101 and 151 MHz, recorded with complete proton decoupling) spectra were obtained with samples dissolved in CDCl₃ or DMSO-d6 with the residual solvent signals used as internal references: 7.26 ppm for CHCl₃, and 2.50 ppm for (CD₃)(CD₂H)S(O) regarding ¹H NMR experiments; 77.2 ppm for CDCl₃ and 39.4 ppm for (CD₃)₂S(O) concerning ¹³C NMR experiments. Chemical shifts (65) are given in ppm to the nearest .01 (1H) or .1 ppm (¹³C). The coupling constants (J) are given in Hertz (Hz). The signals are reported as follows: (s=singlet, d=doublet, t=triplet, m=multiplet, br=broad). Assignments of ¹H and ¹³C NMR signals were unambiguously achieved with the help of D/H exchange and 2D techniques: COSY, NOESY, HMQC, and HMBC experiments. Systematic indole and pyrazole nomenclatures are used below for the assignment of each spectrum. Flash chromatography was performed on Merck silica gel (40.63 m) with the indicated solvent system using gradients of increasing polarity in most cases (Merck KGaA -Darmstadt, Germany). The reactions were monitored by analytical thin-layer chromatography (Merck pre-coated silica gel 60 F254 TLC plates, .25-mm layer thickness). Compounds were visualized on TLC plates by both UV radiation (254 and 365 nm). All solvents for absorption and fluorescence experiments were of spectroscopic grade.

### Methyl Carbamimidothioate Sulfate (2)

To a mixture of thiourea (15.20 g, 200.00 mmol) and water (7 mL) was added Me₂SO₄ (13.80 g, 110.00 mmol) dropwise. After addition, the reaction mixture was heated under reflux for 1 h, cooled to r.t, and ethanol 95% (20 mL) was added. The resulting mixture was cooled to 0° C., filtered, and the filter cake was washed with cold ethanol (10 mL). The white solid was dried over phosphorus pentoxide to afford 2 (23 g, 82%), which was used for the next step without any further purification. Mp: 233.235° C. (EtOH); ¹H NMR (400 MHz, DMSO-d6) δ (ppm) 8.82 (brs, D₂O exchang., 4H), 2.58 (s, 3H).

4-amino-2-(methylthio)pyrimidine-5-carbonitrile (3)

To a solution of 2 (10 g, 3.6 mol) and abs. ethanol (65 mL) was added 2-(ethoxymethylene)malononitrile (8.79 g, 7.2 mol) at 0° C. and triethylamine (12 mL, 8.64 mol). The mixture was stirred at room temperature for 2 hours. After completion of the reaction the resulting precipitate was filtered and washed with ethanol to give the title compound 3 (5.4 g, 89%). ¹H-NMR and ¹³C-NMR are in agreement with the previous reported data (WO2019/126638, 2019, A1).

4-amino-2-(methylsulfonyl)pyrimidine-5-carbonitrile (4)

To a solution of 4-amino-2-(methylthio)pyrimidine-5-carbonitrile (500 mg, 3.01 mmol, 3) in dry CH₂Cl₂ (30 mL), was added m-chloroperbenzoic acid (1.7 g, 9.91 mmol) and the resulting suspension was stirred at rt for 3 hrs. After completion of the reaction, THF was added (20 mL) and the resulting precipitate was filtered and washed with THF. The filtrate was evaporated to dryness and triturated with ether (20 mL) The yellow precipitate was filtered and washed with ether to give the title compound 4 (351 mg, 59%). m.p.: 211.214° C. (EtOAc); ¹H NMR (600 MHz, DMSO-d6) δ (ppm) 8.88 (brs, D₂O exchang., 1H), 8.82 (s, 1H), 8.43 (brs, D₂O exchang., 1H), 3.31 (s, 3H); ¹³C NMR (151 MHz, DMSO-d6) δ (ppm) 166.8, 162.9, 162.4, 114.2, 91.9, 38.6.

4-amino-2-(bis(2-hydroxyethyl)amino)pyrimidine-5-carbonitrile (5α)

To a solution of 4-amino-2-(methylthio)pyrimidine-5-carbonitrile (1.5 g, 7.57 mmol, 4) in dry THF (10 mL), was added diethanolamine (800 mg, 7.6 mmol) and the resulting solution was stirred at rt for 14 hrs. After completion of the reaction, THF was vacuum evaporated and the residue was purified by column chromatography (silica gel, CH₂Cl₂/MeOH: 50/1.20/1) to provide the title compound 5 (532 mg, 52%). M.p.: 142.143° C. ¹H NMR (600 MHz, DMSO-d₆) δ (ppm) 8.24 (s, 1H), 7.13 (br, s, 2H), 4.72 (br, s, 1H), 4.69 (br, s, 1H), 3.68-3.61 (m, 4H), 3.57 (m, 4H); ¹³C NMR (151 MHz, DMSO-d₆) δ (ppm) 163.1, 162.0, 161.6, 117.9, 78.6, 59.2, 51.1.

4-amino-2-(cyclopropylamino)pyrimidine-5-carbonitrile (5b)

This compound was synthesized with an analogous procedure as described for 5α, using cyclopropylamine as the proper amine of the reaction. Yield: 80%, M.p.: 206° C. (MeOH); ¹H NMR (600 MHz, DMSO-d6) δ (ppm) 8.29 (minor) (s, .5H), 8.15 (major) (s, 1H), 7.66 (major) (brs, D₂O exchang., 1H), 7.48 (minor) (brs, D₂O exchang., .5H), 7.23 (major) (brs, D₂O exchang., 2H), 7.04 (minor) (brs, D₂O exchang., 1H), 2.76 (minor+major) (m, 1.5H), .64 (minor+major) (m, 3H), .49 (minor+major) (brs, 3H); ¹³C NMR (151 MHz, DMSO-d6) δ (ppm) 163.3, 162.3, 161.5, 154.3, 153.2, 117.4, 114.9, 79.6, 77.9, 23.8, 22.3, 6.2, 3.7.

4-amino-2-((3-(dimethylamino)propyl)amino)pyrimidine-5-carbonitrile (5d)

This compound was synthesized with an analogous procedure as described for 5α, using -(dimethylamino)propyl)amine as the proper amine of the reaction. Yield: 50%; M.p.: 147° C. (CH₂Cl₂/Et₂O); ¹H NMR (600 MHz, CDCl₃) δ (ppm) 8.25 (minor) (s, .7H), 8.16 (major) (s, 1H), 7.26 (minor+major) (brs, D₂O exchang., .9H), 6.91 (minor+major) (brs, D₂O exchang., 1.7H), 5.54 (minor+major) (brs, D₂O exchang., 1.7H) 3.46 (minor+major) (m, 3.4H), 2.43 (minor+major) (m, 3.4H), 2.27 (major) (s, 6H), 2.25 (minor) (s, 4.2), 1.77 (minor+major) (m, 3.4H); ¹³C NMR (151 MHz, CDCl₃) δ (ppm) 163.5, 162.9, 162.1, 161.8, 161.3, 117.1, 80.3, 78.8, 57.9, 57.0, 45.4, 45.2, 40.6, 39.8, 29.7, 25.9.

(R)-4-amino-2-((1-hydroxybutan-2-yl)amino)pyrimidine-5-carbonitrile (5e)

This compound was synthesized with an analogous procedure as described for 5α, using R-(−)-2-amino-1-butanol as the proper amine of the reaction. Yield: 50%; ¹H NMR (400 MHz, Methanol-d₄) δ 8.17 (minor) (s, 1H), 8.12 (major) (s, 1.5H), 3.99 (minor+major) (s, 2.5H), 3.65.3.49 (minor+major) (m, 5H), 1.85.1.63 (minor+major) (m, 2.5H), 1.54 (minor+major) (m, 2.5H), .98 (m, 7.5H). ¹³C NMR (151 MHz, CDCl₃) δ (ppm) 163.2, 162.5, 161.5, 161.0, 160.6, 160.3, 117.3, 66.2, 65.8, 55.4, 53.2, 24.6, 23.1, 10.7, 10.0.

N-(3-((7-(bis(2-hydroxyethyl)amino)pyrimido[4,5-d]pyrimidin-4-yl)amino)-4-methylphenyl)-3-(trifluoromethyl)benzamide (7α, also referred to herein as CEM452)

To a suspension of compound 5α (240 mg, 1.08 mmol) in dry toluene (50 mL) was added N,N-dimethylformamide-dimethylacetal (142.9 L, 1.08 mmol) and the resulting mixture was stirred at 110° C. for 4 hrs. After completion of the reaction, the volatiles were vacuum evaporated and the oily residue was purified by column chromatography (silica gel, CH₂Cl₂/MeOH: 50/1.20/1) to afford pure 6α (170 mg) as a mixture of E/Z isomers. Without any further purification, to a solution of 6α in glacial acetic acid (2 mL) was added amine 11b (179.8, 0.60 mmol). The resulting mixture was stirred under reflux for 2.5 hrs, and after completion of the reaction, acetic acid was vacuum evaporated. The residue was dissolved in ethanol 95% and to this solution was added dropwise a cold 20% aq. NaOH solution (2 mL). The reaction mixture was stirred at rt overnight, the volatiles were removed in vacuo and the resulting crude was purified by column chromatography on silica gel eluted with CH₂Cl₂/MeOH: 20/1.15/1.12/1 to afford 7α (101 mg, 17%). ¹H NMR (600 MHz, DMSO-d₆) δ (ppm) 10.48 (s, 1H), 9.98 (s, 1H), 9.53 (s, 1H), 8.37 (s, 1H), 8.30 (s, 1H), 8.27 (d, J=7.9 Hz, 1H), 7.96 (d, J=7.8 Hz, 1H), 7.82-7.76 (m, 2H), 7.64 (dd, J=8.3, 2.2 Hz, 1H), 7.31 (d, J=8.3 Hz, 1H), 4.82 (t, J=5.3 Hz, 1H), 4.79 (t, J=5.3 Hz, 1H), 3.84 (t, J=5.6 Hz, 2H), 3.79 (t, J=5.6 Hz, 2H), 3.70.3.61 (m, 4H), 2.17 (s, 3H). ¹³C NMR (151 MHz, DMSO-d₆) δ (ppm) 163.8, 163.4, 162.3, 162.2, 159.6, 158.4, 137.0, 135.7, 131.7, 130.4, 129.7, 129.2, 129.0, 128.0, 124.1, 123.0, 119.6, 118.9, 99.6, 58.9, 58.4, 51.0, 17.4.

N-(3-((7-(bis(2-hydroxyethyl)amino)pyrimido[4,5-d]pyrimidin-4-yl)amino)-4-methylphenyl)-4-(trifluoromethyl)benzamide (7b, also referred to herein as CEM448)

This compound was synthesized with an analogous procedure as described for 7α, using 11α as the proper amine of the reaction. Yield: 20%. ¹H NMR (600 MHz, DMSO-d₆) δ (ppm) 10.48 (s, 1H), 9.97 (s, 1H), 9.53 (s, 1H), 8.37 (s, 1H), 8.15 (d, J=8.1 Hz, 2H), 7.92 (d, J=8.2 Hz, 2H), 7.81 (d, J=2.2 Hz, 1H), 7.63 (dd, J=8.2, 2.2 Hz, 1H), 7.31 (d, J=8.3 Hz, 1H), 4.83 (t, J=5.3 Hz, 1H), 4.79 (t, J=5.3 Hz, 1H), 3.83 (t, J=5.6 Hz, 2H), 3.78 (t, J=5.6 Hz, 2H), 3.70.3.61 (m, 4H), 2.17 (s, 3H). ¹³C NMR (151 MHz, DMSO-d₆) δ (ppm) 164.2, 163.4, 162.2, 162.1, 159.5, 158.4, 138.6, 137.0, 136.4, 131.4, 131.2, 130.5, 130.4, 128.5, 125.3, 125.3, 124.7, 122.9, 119.5, 118.8, 99.5, 58.8, 58.3, 51.0, 17.4.

N-(3-((7-(bis(2-hydroxyethyl)amino)pyrimido[4,5-d]pyrimidin-4-yl)amino)-4-methylphenyl)-isonicotinamide (7c, also referred to herein as CEM446)

This compound was synthesized with an analogous procedure as described for 7α, using 11c as the proper amine of the reaction. Yield: 19%. ¹H NMR (600 MHz, DMSO-d6) 6 (ppm) 10.51 (s, 1H), 9.97 (s, 1H), 9.53 (s, 1H), 8.79 (d, J=5.1 Hz, 2H), 8.37 (s, 1H), 7.86 (d, J=5.1 Hz, 2H), 7.80 (d, J=2.2 Hz, 1H), 7.63 (dd, J=8.3, 2.2 Hz, 1H), 7.32 (d, J=8.3 Hz, 1H), 4.83 (t, J=5.3 Hz, 1H), 4.79 (t, J=5.3 Hz, 1H), 3.83 (t, J=5.6 Hz, 2H), 3.78 (t, J=5.6 Hz, 2H), 3.71.3.61 (m, 4H), 2.17 (s, 3H). ¹³C NMR (151 MHz, DMSO-d₆) δ (ppm) 163.8, 163.4, 162.2, 162.1, 159.5, 158.4, 150.2, 141.8, 136.8, 136.5, 130.7, 130.4, 121.4, 119.5, 118.8, 99.5, 58.8, 58.3, 51.0, 17.94.

N-(3-((7-(cyclopropylamino)pyrimido[4,5-d]pyrimidin-4-yl)amino)-4-methylphenyl)-3-(trifluoromethyl)benzamide (7d, also referred to herein as CEM451)

To a suspension of compound 5b (175 mg, 1.00 mmol) in dry toluene (20 mL) was added N,N-dimethylformamide-dimethylacetal (142.9 L, 1.08 mmol) and the resulting mixture was stirred at 110° C. for 4 hrs. After completion of the reaction, the volatiles were vacuum evaporated and the oily residue was purified by column chromatography (silica gel, cyclohexane/EtOAc: 1/1) to afford pure 6b (160 mg) as a mixture of E/Z isomers. Without any further purification, to a solution of 6b in glacial acetic acid (2 mL) was added amine 11b (179.8, 0.60 mmol). The resulting mixture was stirred under reflux for 1.5 hrs, and after completion of the reaction, acetic acid was vacuum evaporated. The residue was recrystallized in methanol to afford 7d (150 mg, 31%). ¹H NMR (600 MHz, DMSO-d₆) δ (ppm) 10.48 (s, 1H), 9.91 (s, 1H), 9.48 (s, 1H), 8.38 (s, 1H), 8.30 (s, 1H), 8.27 (d, J=8.0 Hz, 1H), 8.08 (s, 1H), 7.96 (d, J=7.8 Hz, 1H), 7.78 (t, J=7.4 Hz, 2H), 7.63 (d, J=8.3 Hz, 1H), 7.31 (d, J=8.3 Hz, 1H), 2.89 (s, 1H), 2.18 (s, 3H), .75 (m, 2H), .57 (m, 2H). ¹³C NMR (151 MHz, DMSO-d₆) δ (ppm) 164.5, 163.9, 162.0, 159.5, 158.5, 137.0, 136.5, 135.7, 131.8, 130.4, 129.7, 129.3, 129.0, 128.1, 124.9, 124.2, 123.1, 119.6, 118.8, 100.1, 24.0, 17.5, 6.2.

N-(3-((7-(cyclopropylamino)pyrimido[4,5-d]pyrimidin-4-yl)amino)-4-methylphenyl)-4-(trifluoromethyl)benzamide (7e, also referred to herein as CEM483)

This compound was synthesized with an analogous procedure as described for 7d, using 11a as the proper amine of the reaction. Yield: 34%. ¹H NMR (600 MHz, DMSO-d6) 6 (ppm) 10.51 (s, 1H), 9.94 (s, 1H), 9.48 (s, 1H), 8.39 (s, 1H), 8.30 (s, 1H), 8.27 (d, J=7.7 Hz, 1H), 8.14 (s, 1H), 7.97 (d, J=7.5 Hz, 1H), 7.79 (s, 1H), 7.78 (t, J=7.7 Hz, 1H), 7.63 (d, J=7.0 Hz, 1H), 7.31 (d, J=8.0 Hz, 1H), 2.88 (s, 1H), 2.17 (s, 3H), .75 (m, 2H), .56 (m, 2H). ¹³C NMR (151 MHz, DMSO-d₆) δ (ppm) 164.5, 163.9, 162.1, 159.5, 158.5, 137.1, 136.6, 135.7, 131.9, 130.5, 129.8, 129.3, 129.1, 128.2, 124.9, 124.2, 123.1, 119.6, 118.9, 100.2, 24.1, 17.5, 6.3.

N-(3-((7-(cyclopropylamino)pyrimido[4,5-d]pyrimidin-4-yl)amino)-4-methylphenyl)-isonicotinamide (7f, also referred to herein as CEM449)

This compound was synthesized with an analogous procedure as described for 7d, using 11c as the proper amine of the reaction. Yield: 30%. ¹H NMR (600 MHz, DMSO-d6) 6 (ppm) 10.51 (s, 1H), 9.95 (s, 1H), 9.47 (s, 1H), 8.78 (d, J=5.1 Hz, 2H), 8.39 (s, 1H), 8.11 (s, 1H), 7.88.7.83 (m, 2H), 7.79 (s, 1H), 7.62 (d, J=8.3 Hz, 1H), 7.31 (d, J=8.3 Hz, 1H), 2.90 (m, 1H), 2.17 (s, 3H), .77-.73 (m, 2H), .57 (m, 2H). ¹³C NMR (151 MHz, DMSO-d₆) δ (ppm) 164.4, 163.8, 161.8, 159.4, 158.4, 150.2, 141.8, 136.8, 136.5, 130.6, 130.4, 121.5, 119.5, 118.8, 97.8, 24.0, 21.0, 17.4, 6.2.

N-(3-((7-(cyclopropylamino)pyrimido[4,5-d]pyrimidin-4-yl)amino)-4-methylphenyl)-2-morpholinoisonicotinamide (7g, also referred to herein as CEM484)

This compound was synthesized with an analogous procedure as described for 7d, using 17α as the proper amine of the reaction. Yield: 31%. ¹H NMR (600 MHz, DMSO-d6) δ 10.32 (s, 1H), 9.89 (s, 1H), 9.48 (s, 1H), 8.38 (s, 1H), 8.28 (dd, J=5.1, 0.7 Hz, 1H), 8.09 (d, J=4.3 Hz, 1H), 7.76 (d, J=2.2 Hz, 1H), 7.61 (d, J=2.2 Hz, 1H), 7.30 (d, J=8.4 Hz, 1H), 7.25 (t, J=1.1 Hz, 1H), 7.12 (dd, J=5.1, 1.3 Hz, 1H), 3.76.3.71 (m, 4H), 3.55.3.51 (m, 4H), 2.90 (s, 1H), 2.17 (s, 3H), .75 (m, 2H), .57 (m, 2H). ¹³C NMR (151 MHz, DMSO-d₆) δ (ppm) 164.9, 164.3, 162.5, 159.9, 158.9, 148.8, 144.2, 137.4, 137.0, 131.0, 130.9, 120.1, 119.3, 111.6, 105.3, 100.6, 66.4, 45.6, 24.5, 17.93, 6.7.

N-(3-((7-(cyclopropylamino)pyrimido[4,5-d]pyrimidin-4-yl)amino)-4-methylphenyl)-2-(dimethylamino)isonicotinamide (7h, also referred to herein as CEM485)

This compound was synthesized with an analogous procedure as described for 7d, using 17b as the proper amine of the reaction. Yield: 41%. ¹H NMR (600 MHz, DMSO-d₆) δ (ppm) 10.29 (s, 1H), 9.89 (s, 1H), 9.47 (s, 1H), 8.38 (s, 1H), 8.23 (d, J=5.2 Hz, 1H), 8.08 (d, J=4.2 Hz, 2H), 7.76 (d, J=2.2 Hz, 1H), 7.62 (dd, J=8.4, 2.3 Hz, 1H), 7.30 (d, J=8.3 Hz, 1H), 7.05 (t, J=1.1 Hz, 1H), 7.00 (dd, J=5.1, 1.4 Hz, 1H), 3.09 (s, 6H), 2.89 (s, 1H), 2.17 (s, 3H), .79-.73 (m, 2H), .57 (m, 2H). ¹³C NMR (151 MHz, DMSO-d₆) δ (ppm) 165.2, 164.9, 162.5, 160.1, 160.0, 159.8, 148.7, 144.9, 143.8, 137.4, 137.0, 130.9, 120.1, 119.3, 109.5, 104.2, 67.5, 38.2, 25.6, 24.5, 17.9, 6.7.

N-(4-methyl-3-((7-(methylthio)pyrimido[4,5-d]pyrimidin-4-yl)amino)phenyl)-3-(trifluoromethyl)benzamide (7i, also referred to herein as CEM443)

This compound was synthesized with an analogous procedure as described for 7d, using 11b as the proper amine of the reaction. Yield: 37%. ¹HNMR (600 MHz, DMSO-d6) 6 (ppm) 10.50 (s, 1H), 10.44 (s, 1H), 9.74 (s, 1H), 8.29 (s, 1H), 8.26 (d, J=7.9 Hz, 1H), 7.96 (d, J=7.8 Hz, 1H), 7.83 (d, J=2.2 Hz, 1H), 7.78 (t, J=7.8 Hz, 1H), 7.64 (dd, J=8.3, 2.3 Hz, 1H), 7.34 (d, J=8.3 Hz, 1H), 2.62 (s, 3H), 2.18 (s, 3H). ¹³C NMR (151 MHz, DMSO-d6) δ (ppm) 175.6, 163.9, 162.7, 162.0, 160.0, 157.6, 137.1, 135.9, 135.7, 131.8, 131.6, 131.2, 130.6, 130.5, 129.7, 128.1, 124.2, 119.4, 119.3, 109.0, 107.7, 104.2, 17.4, 13.8.

N-(4-methyl-3-((7-(methylthio)pyrimido[4,5-d]pyrimidin-4-yl)amino)phenyl)-4-(trifluoromethyl)benzamide (7j, also referred to herein as CEM447)

This compound was synthesized with an analogous procedure as described for 7d, using 11a as the proper amine of the reaction. Yield: 40%. ¹HNMR (600 MHz, DMSO-d6) 6 (ppm) 10.49 (s, 1H), 10.44 (s, 1H), 9.74 (s, 1H), 8.15 (d, J=8.3 Hz, 2H), 7.91 (d, J=8.3 Hz, 2H), 7.85 (s, 1H), 7.63 (d, J=8.3 Hz, 1H), 7.33 (d, J=8.3 Hz, 1H), 2.62 (s, 3H), 2.18 (s, 3H). ¹³C NMR (151 MHz, DMSO-d₆) δ (ppm) 175.6, 164.3, 162.7, 162.0, 159.9, 157.6, 138.6, 137.1, 135.9, 131.6, 131.4, 131.2, 130.6, 130.5, 128.5, 125.4, 125.3, 124.8, 122.9, 119.3, 119.2, 104.2, 17.4, 13.8.

N-(4-methyl-3-((7-(methylthio)pyrimido[4,5-d]pyrimidin-4-yl)amino)phenyl)isonicotinamide (7k, also referred to herein as CEM441)

This compound was synthesized with an analogous procedure as described for 7d, using 11c as the proper amine of the reaction. Yield: 39%. ¹HNMR (600 MHz, DMSO-d₆) δ (ppm) 10.52 (s, 1H), 10.45 (s, 1H), 9.73 (s, 1H), 8.78 (d, J=2.2 Hz, 2H), 8.58 (s, 1H), 7.86 (d, J=2.2 Hz, 2H), 7.83 (s, 1H), 7.63 (dd, J=8.3, 2.2 Hz, 1H), 7.33 (d, J=8.4 Hz, 1H), 2.61 (s, 3H), 2.18 (s, 3H). ¹³C NMR (101 MHz, DMSO-d₆) δ (ppm) 175.6, 166.3, 163.9, 162.8, 162.0, 159.9, 157.7, 152.8, 150.3, 141.9, 137.0, 136.2, 130.7, 121.6, 119.3, 119.2, 104.3, 17.5, 13.9.

N-(3-((7-((3-(dimethylamino)propyl)amino)pyrimido[4,5-d]pyrimidin-4-yl)amino)-4-methylphenyl)-3-(trifluoromethyl)benzamide (71, also referred to herein as CEM481)

This compound was synthesized with an analogous procedure as described for 7d, using 11b as the proper amine of the reaction. Yield: 30%. ¹H NMR (600 MHz, DMSO-d₆) δ (ppm) 10.49 (s, 1H), 9.99 (s, 1H), 9.53 (s, 1H), 8.39 (s, OH), 8.29 (s, 1H), 8.26 (d, J=8.0 Hz, 1H), 8.09 (d, J=6.4 Hz, 1H), 7.97 (d, J=7.8 Hz, 1H), 7.82 (s, 1H), 7.79 (t, J=7.8 Hz, 1H), 7.60 (d, J=8.3 Hz, 1H), 7.31 (d, J=8.3 Hz, 1H), 3.45 (t, J=7.4 Hz, 2H), 2.75 (s, 6H), 2.17 (s, 3H), 1.95 (t, J=7.4 Hz, 2H), 1.26.1.22 (m, 2H). ¹³C NMR (151 MHz, DMSO-d₆) δ 163.82, 163.64, 163.27, 162.00, 159.46, 159.12, 136.98, 136.49, 136.48, 135.66, 131.75, 130.44, 130.31, 129.62, 129.21, 129.00, 128.01, 127.99, 124.81, 124.16, 124.13, 123.00, 119.63, 118.85, 99.98, 66.42, 55.45, 54.63, 52.05, 42.27, 40.06, 37.84, 23.94, 17.42, 7.12.

(R)—N-(3-((7-((1-hydroxybutan-2-yl)amino)pyrimido[4,5-d]pyrimidin-4-yl)amino)-4-methylphenyl)-3-(trifluoromethyl)benzamide (7m, also referred to herein as CEM482)

This compound was synthesized with an analogous procedure as described for 7d, using 11b as the proper amine of the reaction. Yield: 37%. ¹H NMR (600 MHz, CD₃OD) 6 (ppm) 9.33 (s, 1H), 8.34 (s, 1H), 8.24 (s, 1H), 8.18 (d, J=2.2 Hz, 1H), 7.87 (d, J=2.2 Hz, 1H), 7.80 (d, J=2.2 Hz, 1H), 7.71 (t, J=7.8 Hz, 1H), 7.59 (dd, J=8.3, 2.3 Hz, 1H), 7.32 (d, J=2.2 Hz, 1H), 4.10.4.04 (m, 1H), 3.68 (d, J=5.2 Hz, 2H), 2.25 (s, 3H), 1.83.1.71 (m, 2H), 1.68.1.56 (m, 1H), 1.01 (t, J=7.4 Hz, 3H). ¹³C NMR (151 MHz, CD₃OD) 6 (ppm) 165.6, 163.9, 163.6, 161.4, 158.6, 136.9, 135.9, 131.5, 130.9, 130.7, 130.5, 130.2, 129.2, 127.9, 124.7, 124.1, 123.1, 119.9, 119.8, 63.1, 48.0, 23.6, 22.7, 16.38, 9.47.

N-(4-methyl-3-nitrophenyl)-4-(trifluoromethyl)benzamide (10a)

To a solution of aniline 9 (716 mg, 4.71 mmol)) in dry THF (10 mL), 4-(trifluoromethyl)benzoyl chloride (754 p L, 5.0 mmol) and triethylamine (1.45 mL, 10.4 mmol) were added and the resulting mixture was stirred at room temperature for 2.5 hours. After completion of the reaction, volatiles were removed under reduced pressure and water (100 mL) was added to the crude product. The aqueous mixture was washed with EtOAc (3×100 mL), the organic layers were collected, dried over anh. Na₂SO₄ and concentrated under reduced pressure to afford 1.37 g (90%) of the title compound, which was used for the next step without any further purification. ¹H-NMR and ¹³C-NMR are in agreement with the previous reported data (Lo, W.-S., Hu, W.-P., Lo, H.-P., Chen, C.-Y., Kao, C.-L., Vandavasi, J. K., Wang, J.-J.; Org. Lett. 12, 5570.5572, 2010).

N-(4-methyl-3-nitrophenyl)-3-(trifluoromethyl)benzamide (10b)

This compound was synthesized with an analogous procedure as described for 10α, using 3-(trifluoromethyl)benzoyl chloride as the proper chloride of the reaction. Yield: 80%. ¹H-NMR and ¹³C-NMR are in agreement with the previous reported data (Yang, W., Chen, Y., Zhou, X., Gu, Y., Qian, W., Zhang, F., Han, W., Lu, T., Tang, W.; Eur. J. Med. Chem. 89, 581.596, 2015).

N-(4-methyl-3-nitrophenyl)isonicotinamide (10c)

To a solution of 9 (1.52 g, 10 mmol) and isonicotinic acid (1.231 g, 10 mmol) in anh. DMF (5 mL), EDCI (3.82 g, 20 mmol) was added, and the resulting mixture was stirred at room temperature for 16 hrs. After completion of the reaction, the mixture was diluted with water (50 mL) and extracted with EtOAc (3×40 mL). The organic layer was dried (anh. Na₂SO₄) and evaporated to dryness. The residue was purified by column chromatography (silica gel, CH₂Cl₂/MeOH:20/1) to provide 10c (2.36 g, 73%) (JP51147156, 1978). ¹HNMR (600 MHz, CDCl₃) δ (ppm) 8.81 (d, J=2.2 Hz, 2H), 8.26 (d, J=2.2 Hz, 1H), 7.92 (dd, J=8.5, 2.5 Hz, 1H), 7.73 (d, J=2.2 Hz, 2H), 7.36 (d, J=8.3 Hz, 1H), 2.59 (s, 3H). ¹³CNMR (151 MHz, CDCl₃) δ (ppm) 150.9, 149.3, 141.6, 133.6, 130.1, 124.8, 121.0, 116.4, 50.3, 50.1, 49.9, 49.8, 29.8, 20.1.

N-(3-amino-4-methylphenyl)-4-(trifluoromethyl)benzamide (11a)

A suspension of 10α (210 mg, .65 mmol) and SnCl₂—H₂O (630 mg, 2.79 mmol) in anh. acetone (10 mL) was stirred at room temperature for 12 hrs. After completion of the reaction, the mixture was diluted with water (50 mL), basified with 20% NaOH solution and extracted with EtOAc (3×40 mL). The organic layer was dried (anh. Na₂SO₄) and evaporated to dryness. The residue was purified by column chromatography (silica gel, CH₂Cl₂/MeOH:20/1) to provide 11α (120 mg, 63%). ¹H-NMR and ¹³C-NMR are in agreement with the previous reported data (U.S. Ser. No. 11/352,354, 2022, B2).

N-(3-amino-4-methylphenyl)-3-(trifluoromethyl)benzamide (11b)

This compound was synthesized with an analogous procedure as described for 11a, using 10b as staring material. Yield: 65%. ¹H-NMR and ¹³C-NMR are in agreement with the previous reported data (Huang, W., Sun, X., Li, Y., He, Z., Li, L., Deng, Z., Huang, X., Han, S., Zhang, T., Zhong, J., Wang, Z., Xu, Q., Zhang, J., Deng, X.; J. Med. Chem. 61, 5424.5434, 2018).

N-(3-amino-4-methylphenyl)isonicotinamide(11c)

This compound was synthesized with an analogous procedure as described for 11a, using 10b as staring material. Yield: 54%. ¹H-NMR and ¹³C-NMR are in agreement with the previous reported data (Wrobleski, S. T., Lin, S., Hynes, J. Jr., Wu, H., Pitt, S., Shen, D. R., Zhang, R., Gillooly, K. M., Shuster, D. J., McIntyre, K. W., Doweyko, A. M., Kish, K. F., Tredup, J. A., Duke, G. J., Sack, J. S., McKinnon, M., Dodd, J., Barrish, J. C., Schieven, G. L., Leftheris, K.; Bioorg. Med. Chem. Lett. 18, 2739.2744, 2008).

2-chloro-N-(4-methyl-3-nitrophenyl)isonicotinamide (15)

A suspension of 14 (1.57 g, 10 mmol) in POCl₃ (10 mL) was refluxed under Ar for 6 hrs. After completion of the reaction, the volatiles were vacuum evaporated. The resulting chloride was dissolved in dry THF, under Ar, at 0° C. and to this solution, aniline 9 (1.67 g, 11.0 mmol) and triethylamine (1.81 mL, 13.0 mmol) were added, and the resulting mixture was stirred at room temperature for 6 hours. After completion of the reaction, volatiles were removed under reduced pressure and water (80 mL) was added to the crude product. The aqueous mixture was washed with EtOAc (3×80 mL), the organic layers were collected, dried over anh. Na₂SO₄ and concentrated under reduced pressure. The oily residue was purified by column chromatography (silica gel, CH₂Cl₂/MeOH: 97/3) to afford 1.22 g (42%) of the title compound. ¹H-NMR and ¹³C-NMR are in agreement with the previous reported data (EP1165566, 2003, B1).

N-(4-methyl-3-nitrophenyl)-2-morpholinoisonicotinamide (16a)

A solution of 15 (391 mg, 1.5 mmol) and morpholine (1.3 mL, 15 mmol) in anh. DMF (6 mL) was refluxed for 12 hrs. After cooling, the mixture was vacuum evaporated, extracted with EtOAc-water, the organic layer was dried (anh. Na₂SO₄) and evaporated to dryness. The residue was purified by column chromatography (silica gel, CH₂Cl₂/MeOH 97/3) to provide 16α (243 mg, 52%). ¹H-NMR and ¹³C-NMR are in agreement with the previous reported data (W02003/90912, 2003, A1).

2-(dimethylamino)-N-(4-methyl-3-nitrophenyl)isonicotinamide (16b)

This compound was synthesized with an analogous procedure as described for 16a, using dimethylamine as staring material. The reaction was took place in an autoclave apparatus. Yield: 48%. ¹H NMR (600 MHz, DMSO-d₆) δ (ppm) 10.60 (s, 1H), 8.50 (d, J=2.3 Hz, 1H), 8.24 (dd, J=5.1, 0.8 Hz, 1H), 7.98 (dd, J=8.4, 2.3 Hz, 1H), 7.52.7.48 (m, 1H), 7.06 (t, J=1.2 Hz, 1H), 7.01 (dd, J=5.1, 1.4 Hz, 1H), 3.09 (s, 6H). ¹³C NMR (151 MHz, DMSO-d₆) δ (ppm) 165.2, 159.3, 148.5, 148.3, 142.7, 137.6, 133.0, 127.9, 124.9, 115.7, 108.8, 103.7, 37.67, 19.1.

N-(3-amino-4-methylphenyl)-2-morpholinoisonicotinamide (17a)

A solution of 16α (342 mg, 1 mmol) in absolute ethanol (20 mL) was hydrogenated in the presence of 10% Pd/C, under 1 Atm pressure, at room temperature for 6 hrs. After completion of the reaction, the resulting mixture was filtered through a Celite pad, and the filtrate was evaporated to dryness to provide 17α, practically pure (293 mg, 94%). ¹H-NMR and ¹³C-NMR are in agreement with the previous reported data (U.S. Pat. No. 6,593,333, 2003, B1).

N-(3-amino-4-methylphenyl)-2-(dimethylamino)isonicotinamide (17b)

This compound was synthesized with an analogous procedure as described for 17α, using 16b as staring material. Yield: 96%. ¹H NMR (600 MHz, DMSO-d₆) δ (ppm) 9.92 (s, 1H), 8.20 (d, J=5.1 Hz, 1H), 7.07 (d, J=2.1 Hz, 1H), 7.01 (s, 1H), 6.97 (d, J=8.0 Hz, 1H), 6.87 (d, J=8.0 Hz, 1H), 6.80 (dd, J=8.0, 2.1 Hz, 1H), 4.83 (s, 2H), 3.08 (s, 6H), 2.02 (s, 3H). ¹³C NMR (151 MHz, DMSO-d₆) δ (ppm) 164.3, 159.3, 148.1, 146.5, 143.7, 137.5, 129.6, 117.1, 109.1, 108.9, 106.6, 103.6, 37.7, 16.9.

### Example 3: Combination Study of Hdac Inhibitors and Cem451 with Hut78 Cells

### Synergistic Effects of SAHA and CEM451 (Low Doses)

| Dose of | Effect | Dose of | Effect of |
|---|---|---|---|
| SAHA (μM) | of SAHA | CEM451 (μM) | CEM451 |
| 0.6 | 0.3 | 0.5 | 0.24 |
| 0.9 | 0.59 | 1 | 0.26 |
| 1.2 | 0.84 | 3 | 0.45 |

| | Dose of | Effect of | |
|---|---|---|---|
| Dose of | CEM451 | SAHA + | Combination |
| SAHA (μM) | (μM) | CEM451 | index (CI) |
| 0.6 | 0.5 | 0.58 | 0.764 |
| 0.6 | 1 | 0.66 | 0.705 |
| 0.6 | 3 | 0.83 | 0.531 |
| 0.9 | 0.5 | 0.8 | 0.796 |
| 0.9 | 1 | 0.86 | 0.706 |
| 0.9 | 3 | 0.92 | 0.593 |
| 1.2 | 0.5 | 0.92 | 0.782 |
| 1.2 | 1 | 0.93 | 0.752 |
| 1.2 | 3 | 0.96 | 0.638 |

CI > 1-antagonism; CI = 1-additive effect; CI < 1-synergism.

### Synergistic Effects of SAHA and CEM451 (High Doses)

| Dose of | Effect of | Dose of | Effect of |
|---|---|---|---|
| SAHA (μM) | SAHA | CEM451 (μM) | CEM451 |
| 0.6 | 0.16 | 5 | 0.48 |
| 0.9 | 0.27 | 10 | 0.65 |
| 1.2 | 0.74 | 15 | 0.81 |
| | Dose of | Effect of | |
| Dose of | CEM451 | SAHA + | Combination |
| SAHA (μM) | (μM) | CEM451 | index (CI) |
| 0.6 | 5 | 0.78 | 0.78 |
| 0.6 | 10 | 0.89 | 0.73 |
| 0.6 | 15 | 0.93 | 0.70 |
| 0.9 | 5 | 0.86 | 0.79 |
| 0.9 | 10 | 0.93 | 0.72 |
| 0.9 | 15 | 0.96 | 0.64 |
| 1.2 | 5 | 0.96 | 0.60 |
| 1.2 | 10 | 0.97 | 0.61 |
| 1.2 | 15 | 0.97 | 0.68 |

CI > 1-antagonism; CI = 1-additive effect; CI < 1-synergism.

Synergistic Effects on Combination of Romidepsin and CEM451 with Hut78 cells

| Dose of | | Dose of | |
|---|---|---|---|
| Romidepsin | Effect of | CEM451 | Effect of |
| (μM) | Romidepsin | (μM) | CEM451 |
| 0.001 | 0.1 | 5 | 0.57 |
| 0.0015 | 0.58 | 10 | 0.72 |
| 0.002 | 0.92 | 15 | 0.83 |

| Dose of | Dose of | Effect of | |
|---|---|---|---|
| Romidepsin | CEM451 | Romidepsin + | Combination |
| (μM) | (μM) | CEM451 | index (CI) |
| 0.001 | 5 | 0.83 | 0.877 |
| 0.001 | 10 | 0.93 | 0.75 |
| 0.001 | 15 | 0.95 | 0.752 |
| 0.0015 | 5 | 0.95 | 0.706 |
| 0.0015 | 10 | 0.96 | 0.679 |
| 0.0015 | 15 | 0.96 | 0.679 |
| 0.002 | 5 | 0.96 | 0.965 |
| 0.002 | 10 | 0.96 | 1.045 |
| 0.002 | 15 | 0.97 | 1.032 |

CI > 1-antagonism; CI = 1-additive effect; CI < 1-synergism.

Synergistic Effects on Combination of SAHA and CEM451 with RPMI8226 Multiple Myeloma Cells

| Dose of | Effect of | Dose of | Effect of |
|---|---|---|---|
| SAHA (μM) | SAHA | CEM451 (μM) | CEM451 |
| 0.6 | 0.63 | 5 | 0.47 |
| 0.9 | 0.8 | 10 | 0.71 |
| 1.2 | 0.91 | 15 | 0.81 |

| | | Effect of | |
|---|---|---|---|
| Dose of | Dose of | SAHA + | Combination |
| SAHA (μM) | CEM451 (μM) | CEM451 | index (CI) |
| 0.6 | 5 | 0.79 | 1.084 |
| 0.6 | 10 | 0.89 | 0.96 |
| 0.6 | 15 | 0.94 | 0.815 |
| 0.9 | 5 | 0.89 | 1.01 |
| 0.9 | 10 | 0.96 | 0.72 |
| 0.9 | 15 | 0.97 | 0.706 |
| 1.2 | 5 | 0.96 | 0.792 |
| 1.2 | 10 | 0.98 | 0.644 |
| 1.2 | 15 | 0.99 | 0.508 |

CI > 1-antagonism; CI = 1-additive effect; CI < 1-synergism.

What is claimed is:

1. A compound, or a pharmaceutically acceptable salt thereof, having the formula: 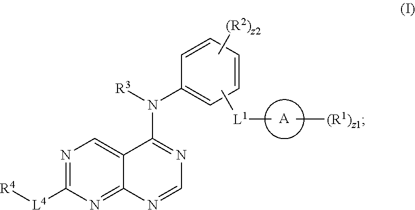
wherein
Ring A is cycloalkyl, heterocycloalkyl, aryl, or heteroaryl;
L¹ is —NR¹⁰C(O)— or —C(O)NR¹⁰—;
L⁴ is a bond, —C(O)—, —C(O)O—, —OC(O)—, —O—, —S—, —NR⁴⁰—, —C(O)NR⁴⁰—,
—NR⁴⁰C(O)—, —NR⁴⁰C(O)O—, —OC(O)NR⁴⁰—, —NR⁴⁰C(O)NR^{40A}—, —S(O)₂—, —NR⁴⁰S(O) ₂—,
—S(O)₂NR⁴⁰—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹,
—OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A} R^{1B},
—OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C},
—SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is independently halogen, —CX²₃, —CHX²₂, —CH₂X², —OCX²₃, —OCH₂X²,
—OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NR^{2C}C(O)NR^{2A}R^{2B},
—N(O)ₘ₂, —NR^{2A}R^{2B}, —C(O)R^{2C}, —C(O)OR^{2C}, —OC(O)R^{2C}, —OC(O)OR^{2C}, —C(O)NR^{2A}R^{2B},
—OC(O)NR^{2A}R^{2B}, —OR^{2D}, —SR^{2D}, —NR^{2A}SO₂R^{2D}, NR^{2A}C(O)R^{2C}, —NR^{2A}C(O)OR^{2C}, —NR^{2A}OR^{2C},
—SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, —CCl₃, —CBr₃, —CF₃, —CI₃, —CH₂Cl, —CH₂Br, —CH₂F, —CH₂I, —CHC1₂,
—CHBr₂, —CHF₂, —CHI₂, —CN, —OH, —NH₂, —COOH, —CONH₂, —OCCl₃, —OCBr₃, —OCF₃, —OCI₃,
—OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂,
—CN, —SOₙ₄R⁴D, —SOv₄NR^{4A}R^{4B}, —NR⁴cNR^{4A}R^{4B}, —ONR^{4A}R^{4B}, —NHC(O)NR^{4A}R^{4B}, N(O)ₘ₄,
—NR^{4A}R^{4B}, —C(O)R^{4C}, —C(O)OR^{4C}, —C(O)NR^{4A}R^{4B}, —OR^{4D}, —SR^{4D}, —NR⁴ASO₂R^{4D}, NR^{4A}C(O)R^{4C}, —NR^{4A}C(O)OR^{4C}, —NR^{4A}OR^{4C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰, R⁴⁰, and R^{40A} are independently hydrogen, —CCl₃, —CBr₃, —CF₃, —CI₃, —CH₂Cl,
—CH₂Br, —CH₂F, —CH₂I, —CHCl₂, —CHBr₂, —CHF₂, —CHI₂, —CN, —OH, —NH₂, —COOH, —CONH₂,
—OCCl₃, —OCBr₃, —OCF₃, —OCI₃, —OCH₂Cl, —OCH₂Br, —OCH₂F, —OCH₂I, —OCHCl₂, —OCHBr₂,
—OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, R^{1D}, R^{2A}, R^{2B}, R^{2C}, R^{2D}, R^{4A}, R^{4B}, R^{4C}, and R^{4D} are independently hydrogen, —CCI₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OC HCl₂, —OCHBr₂, —OCHI₂, —OCHF₂, —OCH₂Cl, —OCH₂Br, —OCH₂J, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
z1 is an integer from 0 to 7;
z2 is an integer from 0 to 4;
X¹, X², and X⁴ are independently —Cl, —Br, —I, or —F;
n1, n2, and n4 are independently an integer from 0 to 4; and
m1, m2, m4, v1, v2, and v4 are independently 1 or 2.

2. The compound of claim 1, having the formula:

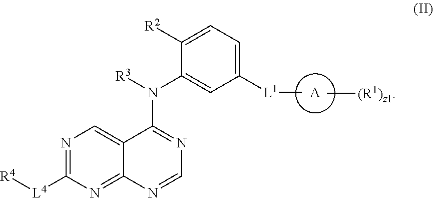

3. The compound of claim 1, having the formula:

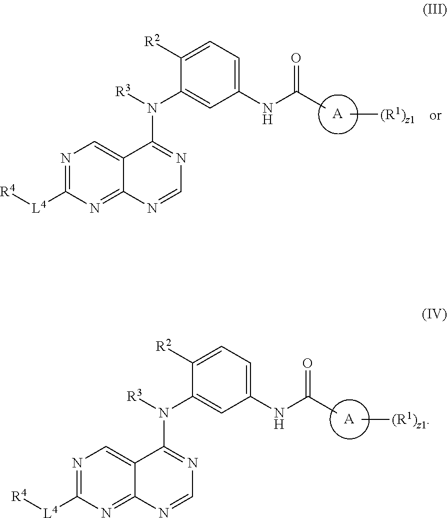

4. The compound of claim 1, wherein Ring A is phenyl or 5 to 6 membered heteroaryl.

5. The compound of claim 1, wherein R¹ is independently halogen, —CF₃, —CHF₂, —CH₂F, —NH₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

6. The compound of claim 1, wherein R¹ is independently —CF₃, —N(CH₃)₂, or unsubstituted morpholinyl.

7. The compound of claim 1, wherein is

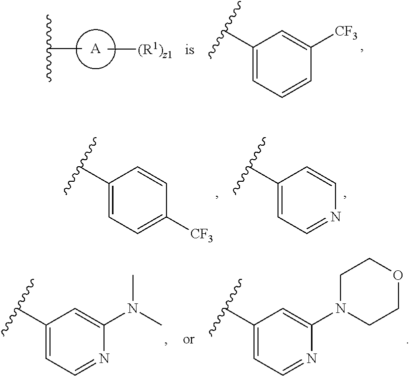

8. The compound of claim 1, wherein R² is independently halogen, —CF₃, —CHF₂, —CH₂F, —NH₂, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

9. The compound of claim 1, wherein R³ is hydrogen or unsubstituted C₁-C₆ alkyl.

10. The compound of claim 1, wherein L⁴ is a bond, —C(O)—,
—C(O)O—, —OC(O)—, —O—, —S—, —NH—, —C(O)NH—, —NHC(O)—, —NHC(O)O—, —OC(O)N H—,
—NHC(O)NH—, —S(O)₂—, —NHS(O)₂—, —S(O)₂NH—, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

11. The compound of claim 1, wherein L⁴ is a bond, —S—, —NH—, or substituted or unsubstituted 2 to 8 membered heteroalkylene.

12. The compound of claim 1, wherein R⁴ is substituted or unsubstituted C₁-C₈ alkyl, substituted or unsubstituted 2 to 8 membered heteroalkyl, or substituted or unsubstituted C₃-C₈ cycloalkyl.

13. The compound of claim 1, wherein -L⁴-R⁴ is —SCH₃,

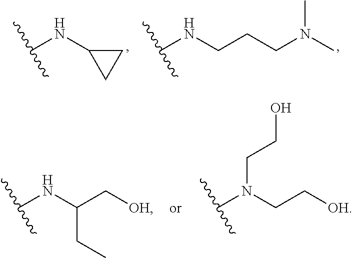

14. The compound of claim 1, having the formula:

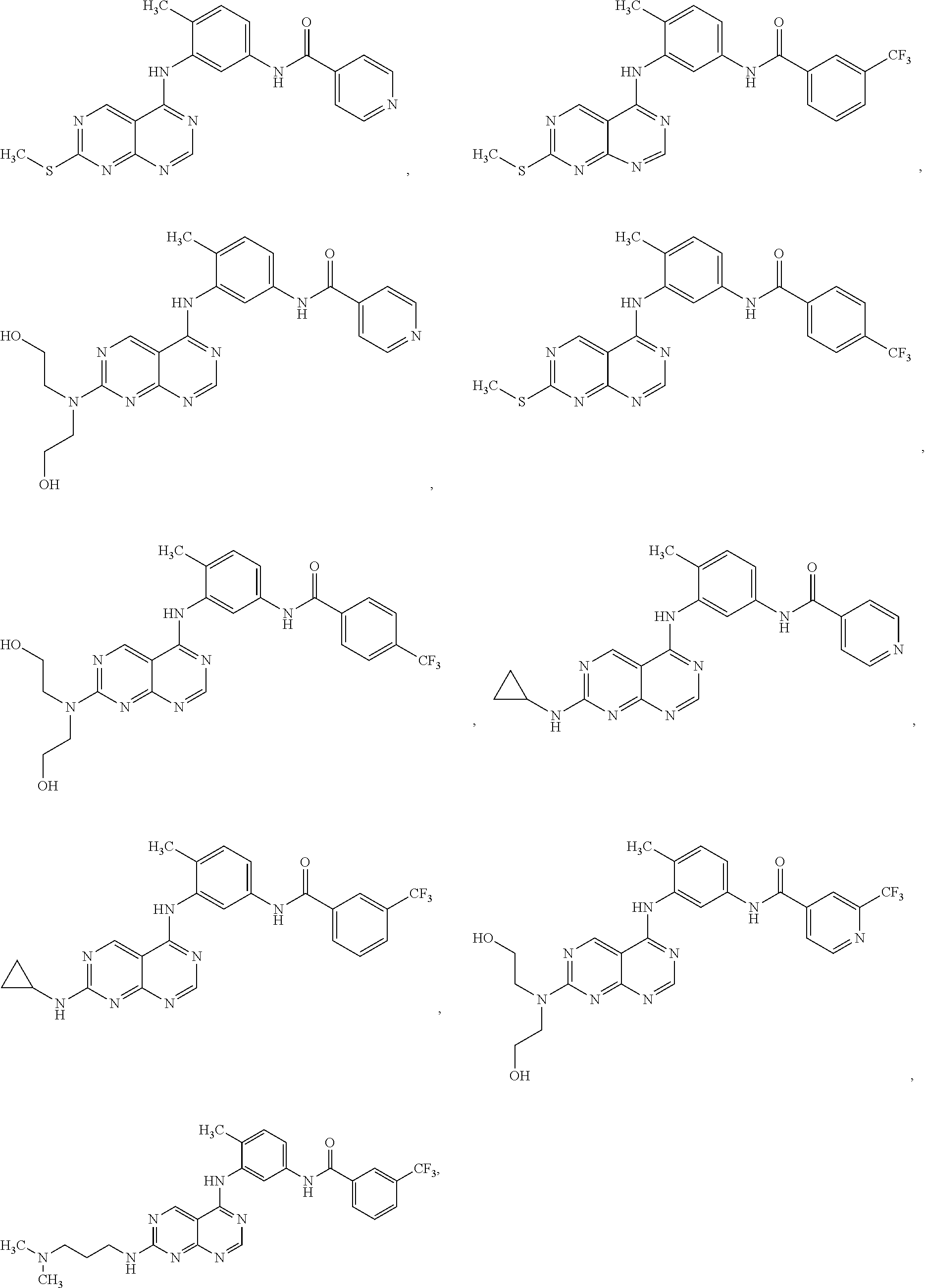

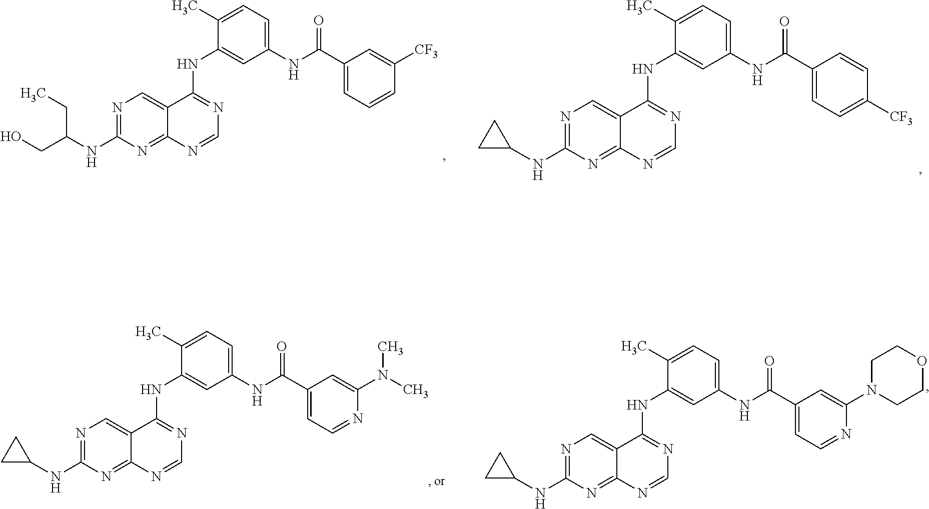 [SMILES: CCC(CO)Nc1ncc2c(Nc3cc(NC(=O)c4cccc(C(F)(F)F)c4)ccc3C)ncnc2n1 ; Cc1ccc(NC(=O)c2ccc(C(F)(F)F)cc2)cc1Nc1ncnc2nc(NC3CC3)ncc12 ; Cc1ccc(NC(=O)c2ccnc(N(C)C)c2)cc1Nc1ncnc2nc(NC3CC3)ncc12 ; Cc1ccc(NC(=O)c2ccnc(N3CCOCC3)c2)cc1Nc1ncnc2nc(NC3CC3)ncc12]

15. A pharmaceutical composition comprising the compound of claim 1 or a pharmaceutically acceptable salt thereof, and a pharmaceutically acceptable excipient.

16. The pharmaceutical composition of claim 15, further comprising an epigenetic inhibitor.

17. The pharmaceutical composition of claim 16, wherein the epigenetic inhibitor is an HDAC inhibitor, and wherein the HDAC inhibitor is vorinostat or romidepsin.

18. A method of treating cancer in a subject in need thereof, said method comprising administering to the subject a therapeutically effective amount of the compound of claim 1, or a pharmaceutically acceptable salt thereof.

19. The method of claim 18, wherein said cancer is lymphoma, leukemia, multiple myeloma, prostate cancer, breast cancer, bladder cancer, liver cancer, lung cancer, ovarian cancer, glioblastoma, pancreatic cancer, renal cancer, head and neck cancer, colon cancer, or melanoma.

20. The method of claim 18, further comprising administering a therapeutically effective amount of an HDAC inhibitor, wherein the HDAC inhibitor is vorinostat or romidepsin.


SEQUENCE LISTING (2 sequences)

SEQ ID NO: 1 - protein, 360 aa, synthetic construct
  REGION 1..360: note = Synthetic polypeptide
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 2 - protein, 364 aa, synthetic construct
  REGION 1..364: note = Synthetic polypeptide
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQALSDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ